(19) United States
(12) Patent Application Publication
(10) Pub. No.: US 2012/0219568 A1
(43) Pub. Date: Aug. 30, 2012

(54) EPIDITHIODIOXOPIPRAZINES AND USES THEREOF IN TREATING CANCER

(75) Inventors: Feiyan Liu, Hangzhou (CN); Kebin Liu, Martinez, GA (US); Ping Wu, Hangzhou (CN)

(73) Assignees: Zhejiang University; Georgia Health Sciences University Research Institute, Inc.

(21) Appl. No.: 13/404,503

(22) Filed: Feb. 24, 2012

Related U.S. Application Data
(60) Provisional application No. 61/446,373, filed on Feb. 24, 2011.

(51) Int. Cl. A61K 39/395; A61P 35/00; C12N 5/09; A61K 31/548; C07D 513/22; A61K 38/19

(52) U.S. Cl. 424/174.1; 544/5; 514/18.9; 435/375; 514/222.8

(57) ABSTRACT

Compositions containing epidithiodioxopiprazines and methods of their use are provided. Epidithiodioxopiprazines can be isolated from natural resources or synthesized de novo. Moreover, epidithiodioxopiprazines, including Verticillin A, are shown to effectively sensitize multiple types of tumor cells to TRAIL-induced apoptosis. In addition, epidithiodioxopiprazines, including Verticillin A, are shown to effectively overcome cancer cell resistance to existing drugs (i.e. Etoposide, Cisplatin, 5-FU and Doxorubicin). Therefore, compositions and methods are provided for use in sensitizing target cancer cells to death receptor- and other anticancer drugs-induced apoptosis. Methods of treating cancer in a subject in need thereof are also provided.

### CROSS REFERENCE TO RELATED APPLICATIONS

This application claims priority to U.S. Provisional Application Ser. No. 61/446,373 filed Feb. 24, 2011, the contents of which are incorporated by reference in its entirety.

### STATEMENT REGARDING FEDERALLY SPONSORED RESEARCH OR DEVELOPMENT

This invention was made with Government Support under Agreement CA133085 awarded to Kebin Liu by the National Institutes of Health. The Government has certain rights in the invention.

### FIELD OF THE INVENTION

The invention is generally related to the field of drug resistance in cancer therapy, more particularly to methods and compositions for overcoming cancer resistance to apoptosis-inducing agonists of death receptors, and existing anticancer drugs, such as etoposide, cisplatin, 5-FU, and doxorubicin.

### BACKGROUND OF THE INVENTION

The ideal cancer therapy should meet two criteria: First, the therapeutic agents have to be effective in killing cancer cells; and second, the treatment needs to have low toxicity, ideally, to be selective for the cancer cells to avoid systemic off-target toxicity (Hall M A and Cleveland J L. Cancer Cell 2007; 12(1):4-6). In reality, cancer cell resistance to chemotherapeutic drugs and high cytotoxicity of chemotherapeutic agents are the two major problems that limit the effectiveness of chemotherapies used to treat human cancer (Longley D B and Johnston P G. J Pathol 2005; 205(2):275-92). Cancer cells may be intrinsically resistant to chemotherapeutic drugs, especially to cytotoxic agents, prior to treatment. Tumors can also acquire resistance during treatment as a result of drug selection pressure on cancer cells. Drug resistance, whether intrinsic or acquired, is believed to account for treatment failure in over 90% patients with metastatic cancer (Longley D B and Johnston P G. J Pathol 2005; 205(2):275-92). Therefore, finding ways to overcome drug resistance may greatly improve the currently disappointing survival rate of patients with cancer. Multiple layers of mechanisms confer cancer cell resistance to chemotherapeutic drugs, however, when it comes to effective eradication of cancer cells by chemotherapies, all roads lead to apoptosis. Essentially all cytotoxic anticancer drugs currently in clinical use or in clinical trials kill cancer cells through inducing apoptosis (Reed J C. Cancer Cell 2003; 3(1):17-22). Thus, tumor cell resistance to apoptosis, whether intrinsic or acquired, represents a major challenge in chemotherapeutic intervention of cancer, especially metastatic cancer.

TNF-related apoptosis-inducing ligand (TRAIL, also known as TNFSF10 or APO2L) is a member of the TNF superfamily. Ever since its discovery in 1995, TRAIL has been under intense study for its obvious potential as a selective anticancer agent in cancer therapy since it preferentially induces apoptosis in tumor cells but not in normal cells (Wiley S R, et al. Immunity 1995; 3(6):673-82; Holoch P A and Griffith T S. Eur J Pharmacol 2009; 625(1-3):63-72). In preclinical mouse models, recombinant TRAIL and agonist TRAIL receptor mAbs exhibited potent tumoricidal activities against TRAIL-sensitive tumors without apparent toxicity (Ashkenazi A, et al. J Clin Invest 1999; 104(2):155-62; Walczak H, et al. Nat Med 1999; 5(2):157-63; Chuntharapai A, et al. J Immunol 2001; 166(8):4891-8). Recombinant TRAIL and agonist TRAIL receptor mAb have been extensively tested in human cancer patients in the clinic (Ichikawa K, et al. Nat Med 2001; 7(8):954-60; Tolcher A W, et al. J Clin Oncol 2007; 25(11):1390-5; Rowinsky E K. J Clin Oncol 2005; 23(36):9394-407). TRAIL-based cancer therapies are now in multiple phase I and phase II clinical trials to treat human cancer (www.clinicaltrials.gov). However, the success of TRAIL-based cancer therapy so far is limited since cancer cells, especially metastatic cancer cells, often exhibit a TRAIL-resistance phenotype (Galligan L, et al. Mol Cancer Ther 2005; 4(12):2026-36; White-Gilbertson S, et al. Oncogene 2009; 28(8):1132-41; Garofalo M, et al. Cancer Cell 2009; 16(6):498-509; Kim S H, et al. Cancer Res 2008; 68(7):2062-4).

To overcome cancer cell resistance to TRAIL-induced apoptosis, various therapeutic agents have been tested for their effectiveness in enhancing TRAIL-induced apoptosis (Rosato R R, et al. Cancer Res 2007; 67(19):9490-500; Rosato R R, et al. Mal Cancer Ther 2003; 2(12):1273-84; Lagneaux L, et al. Exp Hematol 2007; 35(10):1527-37; Ricci M S, et al. Cancer Cell 2007; 12(1):66-80; Nawrocki S T, et al. Cancer Res 2007; 67(14):6987-94; Shankar S, et al. Mol Cancer Ther 2009; 8(6):1596-605). These therapeutic agents have shown great promise in enhancing TRAIL efficacy. However, because the most attractive feature of TRAIL therapy is its tumor selectivity-conferred low toxicity, combining cytotoxic agents with TRAIL may bring back toxicity associated with the therapeutic agents. Therefore, identifying novel TRAIL sensitizers with low toxicity and high sensitization activity is in urgent need for TRAIL-based cancer therapy.

Therefore, it is an object of the invention to provide improved TRAIL sensitizers for use in TRAIL-based cancer therapy. Preferably, the TRAIL sensitizers have low toxicity and high activity.

It is another object of the invention to provide methods for treating cancer using TRAIL in combination with one or more TRAIL sensitizers.

It is another object of the invention to provide methods and compositions for sensitizing tumor cells to TRAIL-induced apoptosis.

It is another object of the invention to provide methods and compositions for overcoming cancer resistance to existing therapeutic drugs including etoposide, cisplatin, 5-FU, and doxorubicin.

It is a further object of the invention to provide methods and compositions for sensitizing cells to apoptosis.

### SUMMARY OF THE INVENTION

Compositions containing one or more epidithiodioxopiprazines and methods of their use are provided. In some embodiments, the epidithiodioxopiprazines sensitize cells, such as cancer cells, to apoptosis. Apoptosis is induced in cancer cells with chemotherapeutics and death receptor agonists; however cancer cells can become resistant to these therapies. Therefore, epidithiodioxopiprazines can be used to sensitize the cancer cells to these therapies and enhance their effects.

In some embodiments, the epidithiodioxopiprazines overcome cancer resistance to TRAIL or increase the efficacy of TRAIL in the treatment of cancer. In a preferred embodiment, one or more epidithiodioxopiprazines sensitizes target cells to TRAIL-induced apoptosis. In other embodiments, one or more epidithiodioxopiprazines increase the efficacy of anti-neoplastic agents, in the treatment of cancer. In preferred embodiments, one or more epidithiodioxopiprazines are shown to decrease cancer resistance to existing therapeutic drugs including etoposide, cisplatin, 5-FU, and doxorubicin, etoposide, cisplatin, 5-FU, and doxorubicin. A preferred epidithiodioxopiprazine is Verticillin A or a derivative or prodrug thereof. Verticillin A is a potent cytotoxin typically isolated from pathogen-infected poisonous mushrooms.

One embodiment provides compositions containing one or more epidithiodioxopiprazines in an amount effective to sensitize target cells to death receptor-induced apoptosis. The compositions can be administered to a subject, preferably a human subject to treat cancer. In some embodiments, the composition contains one or more epidithiodioxopiprazines described by Formulas I-V in an amount effective to sensitize a cancer cell to TRAIL-induced apoptosis. In certain embodiments, the cancer cells to be sensitized have resistance to TRAIL-induced apoptosis. For example, in some embodiments the composition contains Verticillin A. In other embodiments, the composition contains Verticillin B, D, E, or F, 11-deoxyverticillin, 11,11′-dideoxyverticillin, Chaetocin, Gliotoxin, or Chaetomin.

In another embodiment, the composition contains an effective amount of one or more epidithiodioxopiprazines to sensitize target cells to TRAIL-induced apoptosis and an effective amount of a death receptor agonist to induce apoptosis. For example, the death receptor agonist can be TRAIL or an antibody that selectively binds and activates DR4 (TRAIL-R1) or DR5 (TRAIL-R2). In some embodiments, the composition contains Verticillin A in an amount effective to sensitize a TRAIL-resistant cancer cell to TRAIL-induced apoptosis. In further embodiments, the epidithiodioxopiprazine is Verticillin B, D, E, or F, 11-deoxyverticillin, 11,11′-dideoxyverticillin, Chaetocin, Gliotoxin, Chaetomin, or a combination thereof.

Another embodiment provides a method of inducing apoptosis in a target cell. The method includes contacting the target cell with a first composition containing an effective amount of one or more epidithiodioxopiprazines described by Formulas I-V. In some embodiments, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, Chaetomin, or a combination thereof. The method can also involve contacting the target cell with a second composition containing an effective amount of death receptor agonist. In some embodiments, the cell is contacted with the first composition from about 1 minute to about 1 hour before the second composition. In other embodiments, the cell is contacted with the first composition less than 1 minute before the second composition. In preferred embodiments, the target cell expresses DR4 (TRAIL-R1) or DR5 (TRAIL-R2). For example, the target cell can be a cancer cell or a tumor cell. Preferrably, the target cell is resistant to TRAIL-induced apoptosis in the absence of an epidithiodioxopiprazine.

A method of treating cancer in a subject in need thereof is also provided. Some embodiments of the method involve administering to the subject a composition containing a therapeutically effective amount of one or more epidithiodioxopiprazines described by Formulas I-V and a death receptor agonist. In some embodiments, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, Chaetomin, or a combination thereof. Other embodiments of the method involve administering to the subject a first composition comprising a therapeutically effective amount of one or more epidithiodioxopiprazines described by Formulas I-V and a second composition containing a therapeutically effective amount of a death receptor agonist. In one embodiment, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin. For example, the first composition can be administered from about 1 minute to about 1 hour before the second composition. Alternatively, the first composition can be administered less than minute before the second composition. In preferred embodiments, the cancer is resistant to TNF-related apoptosis-inducing ligand (TRAIL) treatment.

The death receptor agonist of the disclosed methods can be a death receptor ligand, such as TRAIL. Alternatively, the death receptor agonist can be an antibody, ligand, or small molecules that selectively binds and activates a death receptor such as DR4 (TRAIL-R1) or DR5 (TRAIL-R2).

### BRIEF DESCRIPTION OF THE DRAWINGS

FIG. 1 shows the chemical structure of Veticillin A.

FIG. 2A is a line graph showing growth rate inhibition (%) of heptoma HepG2 cells as a function of Verticillin A concentration (nM) treatment for 24 h (open circles) or 72 h (closed circles) measured using an MTT assay. FIG. 2B is a bar graph showing heptoma tumor volume (mm³) in athymic mice injected with HepG2 cells 0 days (first set of bars), 7 days (second set bars), 10 days (third set of bars), and 14 days (fourth set of bars) after treatment with control (left bars in each set), 1 mg/kg body weight Verticillin A (middle bars in each set), or 2 mg/kg body weight Verticillin A (right bars in each set). *p<0.05.

FIG. 3A is a line graph showing cell death (%) of SW620 metastatic human colon carcinoma cells as a function of Verticillin A concentration (nM). FIG. 3B is a line graph showing cell death (%) of SW620 cells as a function of TRAIL concentration (ng/ml) alone (open circles) or in combination with overnight pre-treatment with 10 nM Verticillin A (closed circles). FIG. 3C is a line graph showing cell death (%) of SW620 cells as a function of DR5 mAb (ng/ml) (open circles) alone or in combination with overnight pre-treatment with 10 nM Verticillin A (closed circles). FIG. 3D is a bar graph showing cell death (%) of the colon carcinoma cells LS114N (first set of bars), T84 (second set of bars), Colo201 (third set of bars), Colo205 (fourth set of bars), Caco2 (fifth set of bars), and LS174T (sixth set of bars) treated (as in FIG. 3A) with Verticillin A alone (left bars in each set), TRAIL alone (middle bars in each set), or Verticillin A and TRAIL (right bars in each set). FIG. 3E is a bar graph showing cell death (%) of sarcoma cells MC-WST-724 (first set of bars), ovarian carcinoma cells A549 (second set of bars) and mammary carcinoma cells MCF-7 (third set of bars) treated (as in FIG. 3A) with Verticillin A alone (left bars in each set), TRAIL alone (middle bars in each set), or Verticillin A and TRAIL (right bars in each set).

FIG. 4 is a bar graph showing carcinoma tumor volume (mm³) in athymic mice injected with SW620 cells (3×10⁶ cells/mouse) 0 days (first set of bars), 10 days (second set bars), 12 days (third set of bars), 15 days (fourth set of bars), and 17 days (fifth set of bars) after treatment (three days after cell injection) with control (first bar in each set), 0.125 mg/kg Verticillin A (second bar in each set), 100 mg TRAIL (third bar in each set), or Verticillin A and TRAIL (fourth bar in each set). *p<0.05.

FIG. 5 is a line graph showing cell death (%) as a function of FasL concentration for SW620 cells incubated overnight with control (open circle) or 10 nM verticillin A (closed circle) followed by incubation with various concentrations of FasL (ng/ml).

FIG. 6A is a bar graph showing cell death (%) of SW620 cells treated with control (first bar), 20 nM Verticillin A alone (overnight pre-treatment) (second bar), 1 μg/ml Etoposide (third bar), or Verticillin A and Etoposide (fourth bar) for 3 days and measured for cell growth with an MTT assay. FIG. 6B is a bar graph showing cell death (%) of SW620 cells treated with control (first bar), 20 nM Verticillin A alone (overnight pre-treatment) (second bar), 1 μg/ml Cisplatin (third bar), or Verticillin A and Cisplatin (fourth bar) for 3 days and measured for cell growth with an MTT assay. FIG. 6C is a bar graph showing cell death (%) of SW620 cells treated with control (first bar), 20 nM Verticillin A alone (overnight pre-treatment) (second bar), 0.1 μg/ml 5-FU (third bar), or Verticillin A and 5-FU (fourth bar) for 3 days and measured for cell growth with an MTT assay. FIG. 6D is a bar graph showing cell death (%) of SW620 cells treated with control (first bar), 20 nM Verticillin A alone (overnight pre-treatment) (second bar), 0.01 μg/ml Doxorubicin (third bar), or Verticillin A and Doxorubicin (fourth bar) for 3 days and measured for cell growth with an MTT assay. **p<0.01.

FIG. 7A is a bar graph showing cell death (%) of LS411N cells (first set of bars), T84 cells (second set of bars), LS174T cells (third set of bars), and SW620 cells (fourth set of bars) transfected with scrambled siRNA (first bar) or acid ceramidase (A-CDase)-specific siRNA (second bar). FIG. 7B is a bar graph showing cell death (%) of SW620 cells incubated overnight with control (middle bar in each set) or the A-CDase inhibitor LCL85 (first and third bars in each set) and then treated with TRAIL (second and third bars in each set). ** p<0.01.

FIG. 8 is a bar graph showing cell death (%) of SW620 cells incubated with C16 ceramide for 2 h followed by treatment with TRAIL overnight. The tumor cells were then stained with PI and annex V and analyzed by flow cytometry. % apoptotic cells were calculated by the formula [% annexin V⁺ cells of the TRAIL-treated cells-% annexin V⁺ cells of untreated cells].

FIG. 9 is a chart showing the structure of human BNIP3 promoter. The locations of the CpG islands are indicated.

FIG. 10A is bar graph showing Verticillin A-induced cell death (%) in HepG2 cells after treatment with scrambled siRNA or BNIP3 siRNA. Silencing BNIP3 in HepG2 cell decreased the tumor cell sensitivity to Verticillin A-induced cell death. FIG. 10B is bar graph showing Verticillin A-induced cell death (%) in human colon carcinoma SW620 cells after treatment with scrambled siRNA or BNIP3 siRNA. Silencing BNIP3 in HepG2 cell decreased the tumor cell sensitivity to Verticillin A-induced cell death. **p<0.01.

FIGS. 11A and 11B show that Verticillin A up-regulates TET1 in human colon carcinoma cells. FIG. 11A is an acrylamide gel showing that LS411N cells were treated with Verticillin A at the indicated concentrations for 24 h and analyzed for TET1, TET2 and TET3 level by RT-PCR. FIG. 11B shows an acrylamide gel and a bar graph wherein RKO cells were analyzed for TET1 expression level by conventional RT-PCR (top panel) and real-time RT-PCR (bottom panel).

### DETAILED DESCRIPTION OF THE INVENTION

### I. Definitions

The term “co-administration” includes simultaneous and sequential administration. An appropriate time course for sequential administration may be chosen by the physician, according to such factors as the nature of a patient's illness, and the patient's condition.

The term “death receptor” refers to a cell-surface receptor that induces cellular apoptosis once bound by a ligand. Death receptors preferably include tumor necrosis factor (TNF) receptor superfamily members having death domains (e.g., TNFR1, Fas, DR3, DR4, DR5, DR6, and LTβR).

The term “death receptor agonist” refers to a substance that is capable of binding a death receptor on a cell and initiating apoptosis. For example, a “death receptor agonist small molecule” is a compound that is capable of interacting with the death receptor to initiate apoptosis.

The term “inhibit,” “inhibiting,” or “inhibition” refers to a decrease in activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “subject” refers to any individual who is the target of administration. The subject can be a vertebrate, for example, a mammal. Thus, the subject can be a human. The term does not denote a particular age or sex. The term “patient” refers to a subject afflicted with a disease or disorder. The term “patient” includes human and veterinary subjects.

The term “target cell” refers to a cell bearing the targeted death receptor, including, for example, a cell that expresses DR5 or DR4. Preferably, the target cell is an abnormally growing cell or tumor cell.

The term “therapeutically effective” means that the amount of the composition used is of sufficient quantity to ameliorate one or more causes or symptoms of a disease or disorder. Such amelioration only requires a reduction or alteration, not necessarily elimination. For example, a therapeutically effective amount of a composition containing a death receptor agonist is the quantity sufficient to cause apoptosis in one or more target cells. As used herein, the terms “therapeutically effective amount” “therapeutic amount” and “pharmaceutically effective amount” are synonymous. One of skill in the art could readily determine the proper therapeutic amount.

The term “treat” or “treatment” refers to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent,” “preventing,” or “prevention” does not require absolute forestalling of the condition or disease but can also include a reduction in the onset or severity of the disease or condition. For example, in the case of death receptor resistance, to prevent a target cell's resistance to a death receptor agonist is to make the cell less resistant to said agonist.

The terms “Analog” and “Derivative” are used herein interchangeably, and refer to a compound having a structure similar to that a parent compound, but varying from the parent compound by a difference in one or more certain components. The analog or derivative can differ from the parent compound in one or more atoms, functional groups, or substructures, which are replaced with other atoms, groups, or substructures. An analog or derivative can be imagined to be formed, at least theoretically, from the parent compound via some chemical or physical process. The terms analog and derivative encompass compounds which retain the same basic ring structure as the parent compound, but possesses one or more different substituents on the ring(s). The terms analog and derivative also encompasses compounds which possesses a different ring structure from the parent compound which is obtained via chemical modification of the parent compound.

The term “Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “Alkyl”, as used herein, refers to the radical of saturated or unsaturated aliphatic groups, including straight-chain alkyl, alkenyl, or alkynyl groups, branched-chain alkyl, alkenyl, or alkynyl groups, cycloalkyl, cycloalkenyl, or cycloalkynyl (alicyclic) groups, alkyl substituted cycloalkyl, cycloalkenyl, or cycloalkynyl groups, and cycloalkyl substituted alkyl, alkenyl, or alkynyl groups. Unless otherwise indicated, a straight chain or branched chain alkyl has 30 or fewer carbon atoms in its backbone (e.g., C₁-C₃₀ for straight chain, C₃-C₃₀ for branched chain), preferably 20 or fewer, more preferably 10 or fewer, most preferably 6 or fewer. If the alkyl is unsaturated, the alkyl chain generally has from 2-30 carbons in the chain, preferably from 2-20 carbons in the chain, more preferably from 2-10 carbons in the chain. Likewise, preferred cycloalkyls have from 3-20 carbon atoms in their ring structure, preferably from 3-10 carbons atoms in their ring structure, most preferably 5, 6 or 7 carbons in the ring structure.

The terms “alkenyl” and “alkynyl” refer to unsaturated aliphatic groups analogous in length and possible substitution to the alkyls described above, but that contain at least one double or triple bond respectively.

The term “alkyl” includes one or more substitutions at one or more carbon atoms of the hydrocarbon radical as well as heteroalkyls. Suitable substituents include, but are not limited to, halogens, such as fluorine, chlorine, bromine, or iodine; hydroxyl; —NR₁R₂, wherein R₁ and R₂ are independently hydrogen, alkyl, or aryl, and wherein the nitrogen atom is optionally quaternized; —SR, wherein R is hydrogen, alkyl, or aryl; —CN; —NO₂; —COOH; carboxylate; —COR, —COOR, or —CONR₂, wherein R is hydrogen, alkyl, or aryl; azide, aralkyl, alkoxyl, imino, phosphonate, phosphinate, silyl, ether, sulfonyl, sulfonamido, heterocyclyl, aromatic or heteroaromatic moieties, —CF₃; —CN; —NCOCOCH₂CH₂; —NCOCOCHCH; —NCS; and combinations thereof.

The terms “amino” and “amine”, as used herein, are art-recognized and refer to both substituted and unsubstituted amines, e.g., a moiety that can be represented by the general formula:

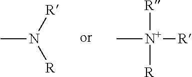

wherein, R, R′, and R″ each independently represent a hydrogen, substituted or unsubstituted alkyl, substituted or unsubstituted alkenyl, substituted or unsubstituted alkynyl, substituted or unsubstituted carbonyl, —(CH₂)ₘ—R′″, or R and R′ taken together with the N atom to which they are attached complete a heterocycle having from 3 to 14 atoms in the ring structure; R′″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. In preferred embodiments, only one of R and R′ can be a carbonyl, e.g., R and R′ together with the nitrogen do not form an imide. In preferred embodiments, R and R′ (and optionally R″) each independently represent a hydrogen atom, substituted or unsubstituted alkyl, a substituted or unsubstituted alkenyl, or —(C₁₋₁₂)ₘ—R′″. Thus, the term ‘alkylamine’ as used herein refers to an amine group, as defined above, having a substituted or unsubstituted alkyl attached thereto (i.e. at least one of R, R′, or R″ is an alkyl group).

The term “carbonyl”, as used herein, is art-recognized and includes such moieties as can be represented by the general formula:

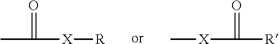

wherein X is a bond, or represents an oxygen or a sulfur, and R represents a hydrogen, a substituted or unsubstituted alkyl, substituted or unsubstituted alkenyl, substituted or unsubstituted alkynyl, —(CH₂)ₘ—R″, or a pharmaceutical acceptable salt, R′ represents a hydrogen, substituted or unsubstituted alkyl, substituted or unsubstituted alkenyl, substituted or unsubstituted alkynyl, or —(CH₂)ₘ—R″; R″ represents a hydroxy group, substituted or unsubstituted carbonyl group, an aryl, a cycloalkyl ring, a cycloalkenyl ring, a heterocycle, or a polycycle; and m is zero or an integer ranging from 1 to 8. Where X is oxygen and R is defines as above, the moiety is also referred to as a carboxyl group. When X is oxygen and R is hydrogen, the formula represents a ‘carboxylic acid’. Where X is oxygen and R′ is hydrogen, the formula represents a ‘formate’. In general, where the oxygen atom of the above formula is replaced by a sulfur, the formula represents a ‘thiocarbonyl’ group. Where X is sulfur and R or R′ is not hydrogen, the formula represents a ‘thioester’. Where X is sulfur and R is hydrogen, the formula represents a ‘thiocarboxylic acid’. Where X is sulfur and R′ is hydrogen, the formula represents a ‘thioformate’. Where X is a bond and R is not hydrogen, the above formula represents a ‘ketone’. Where X is a bond and R is hydrogen, the above formula represents an ‘aldehyde’.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized.

Examples of saturated hydrocarbon radicals include, but are not limited to, methyl, ethyl, n-propyl, isopropyl, n-butyl, t-butyl, isobutyl, sec-butyl, cyclohexyl, (cyclohexyl)methyl, cyclopropylmethyl, and homologs and isomers of, for example, n-pentyl, n-hexyl, n-heptyl, n-octyl. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, and 3-butynyl.

The terms “Alkoxy”, “alkylamino”, and “alkylthio” are used herein in their conventional sense, and refer to those alkyl groups attached to the remainder of the molecule via an oxygen atom, an amino group, or a sulfur atom, respectively.

The term “Alkylaryl”, as used herein, refers to an alkyl group substituted with an aryl group (e.g., an aromatic or hetero aromatic group).

The terms “Heterocycle” or “heterocyclic”, as used herein, refers to a cyclic radical attached via a ring carbon or nitrogen of a monocyclic or bicyclic ring containing 340 ring atoms, and preferably from 5-6 ring atoms, consisting of carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Examples of heterocyclic ring include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents as defined above for alkyl and aryl.

The term “Halogen”, as used herein, refers to fluorine, chlorine, bromine, or iodine.

The term “Pharmaceutically acceptable salt”, as used herein, refer to derivatives of the compounds defined herein, wherein the parent compound is modified by making acid or base salts thereof. Example of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic salts.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704; and “Handbook of Pharmaceutical Salts: Properties, Selection, and Use,” P. Heinrich Stahl and Camille G. Wermuth, Eds., Wiley-VCH, Weinheim, 2002.

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

### II. Compositions

Compositions for treating cancer or tumor cells include one or more active agents. Active agents include epidithiodioxopiprazines, death receptor agonists, and other therapeutic agents, for example anti-inflammatories, anti-infective agents, or anti-neoplastic agents. In some embodiments, compositions contain multiple active agents. In other embodiments, active agents are administered in separate compositions, either simultaneously or at different times.

In some embodiments, the compositons include one or more epidithiodioxopiprazines and optionally an excipient. In some embodiments, the compositons include one or more epidithiodioxopiprazines, one or more death receptor agaonists, and optionally an excipient. In some embodiments, the compositons include one or more epidithiodioxopiprazines, one or more anti-neoplastic agents, and optionally an excipient. In further embodiments, the compositons include one or more epidithiodioxopiprazines, one or more death receptor agaonists, one or more anti-neoplastic agents, and optionally an excipient. In certain embodiments, the compositions can further include additional active agents, for example anti-inflammatories, anti-infective agents.

A. Epidithiodioxopiprazines

A variety of epidithiodioxopiprazines are useful in such compositions and methods. Epidithiodioxopiprazines can be synthesized de novo or isolated from natural resources using methods and techniques known to those of ordinary skill in the art.

1. Structure of Epidithiodioxopiprazines

In one embodiment, the epidithiodioxopiprazine is represented by Formula I:

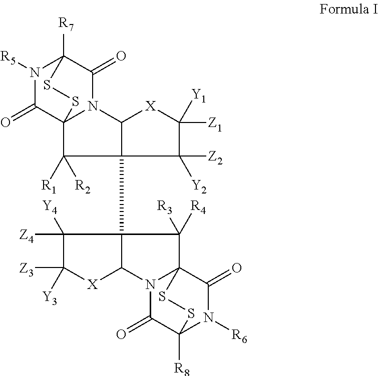

wherein

R₁-R₄ taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or

R₁ is absent and R₂ may be a ketone or a substituted or unsubstituted exocyclic alkylene group, and/or R₃ is absent and R₄ may be a ketone or a substituted or unsubstituted exocyclic alkylene group;

R₅-R₈, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;

Y₁-Y₄ and Z₁-Z₄, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

Y₁ and Y₂, Z₁ and Z₂, Y₃ and Y₄, and/or Z₃ and Z₄, taken together with the atoms to which they are attached, may be C₃-C₂₀ cyclic, substituted C₃-C₂₀ cyclic, heterocyclic, or substituted heterocyclic group; or

Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; or

Z₃ and Z₄ are absent and Y₃ and Y₄, taken together with the atoms to which they are attached may be a π-bond, phenyl, substituted phenyl, aryl, substituted aryl, heteroaryl, substituted heteroaryl, polyaryl, or substituted polyaryl; or

Z₁ is absent and Y₁ may be a ketone or a substituted or unsubstituted exocyclic alkylene group, Z₂ is absent and Y₂ may be a ketone or a substituted or unsubstituted exocyclic alkylene group, Z₃ is absent and Y₃ may be a ketone or a substituted or unsubstituted exocyclic alkylene group, and/or Z₄ is absent and Y₄ may be a ketone or a substituted or unsubstituted exocyclic alkylene group; and

X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;

or a pharmaceutically acceptable salt thereof.

In another embodiment, the epidithiodioxopiprazine is represented by

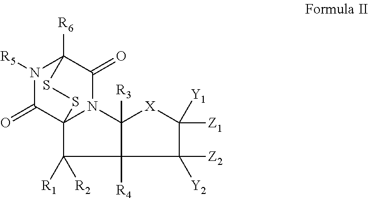

wherein

R₁-R₄ taken independently may be absent, or may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or

R₁ is absent and R₂ may be a ketone, a substituted or unsubstituted exocyclic alkylene group;

R₅ and R₆, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅ and R₆ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;

Y₁-Y₂ and Z₁-Z₂, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

Y₁ and Y₂ and/or Z₁ and Z₂, taken together with the atoms to which they are attached, may be C₃-C₂₀ cyclic, substituted C₃-C₂₀ cyclic, heterocyclic, or substituted heterocyclic group; or

Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; and

X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;

or a pharmaceutically acceptable salt thereof.

In another embodiment, the epidithiodioxopiprazine is represented by Formula III:

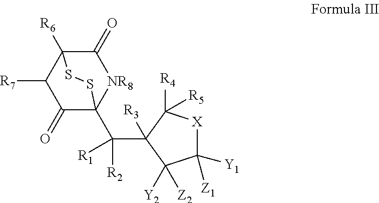

wherein

R₁-R₅ taken independently may be absent, or may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₅ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

R₁ and R₂ and/or R₄ and R₅ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or

R₁ is absent and R₂ may be a ketone, a substituted or unsubstituted exocyclic alkylene group, and/or R₄ is absent and R₅ may be a ketone, a substituted or unsubstituted exocyclic alkylene group;

R₆ and R₇, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₆ and R₇ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;

Y₁-Y₂ and Z₁-Z₂, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

Y₁ and Y₂ and/or Z₁ and Z₂, taken together with the atoms to which they are attached, may be C₃-C₂₀ cyclic, substituted C₃-C₂₀ cyclic, heterocyclic, or substituted heterocyclic group; or

Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; and

X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;

or a pharmaceutically acceptable salt thereof.

In another embodiment, the epidithiodioxopiprazine is represented by Formula IV:

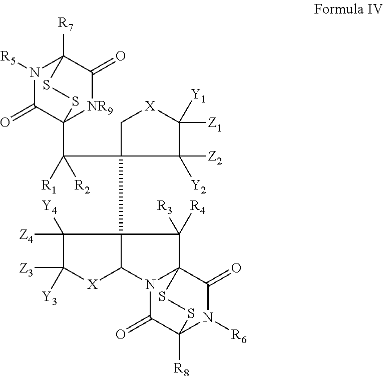

wherein

R₁-R₄ taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or

R₁ is absent and R₂ may be a ketone, a substituted or unsubstituted exocyclic alkylene group, and/or R₃ is absent and R₄ may be a ketone, a substituted or unsubstituted exocyclic alkylene group;

R₅-R₉, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;

Y₁-Y₄ and Z₁-Z₄, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

Y₁ and Y₂, Z₁ and Z₂, Y₃ and Y₄, and/or Z₃ and Z₄, taken together with the atoms to which they are attached, may be C₃-C₂₀ cyclic, substituted C₃-C₂₀ cyclic, heterocyclic, or substituted heterocyclic group; or

Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; or

Z₃ and Z₄ are absent and Y₃ and Y₄, taken together with the atoms to which they are attached may be a π-bond, phenyl, substituted phenyl, aryl, substituted aryl, heteroaryl, substituted heteroaryl, polyaryl, or substituted polyaryl; or

Z₁ is absent and Y₁ may be a ketone, a substituted or unsubstituted exocyclic alkylene group, and/or Z₂ is absent and Y₂ may be a ketone, a substituted or unsubstituted exocyclic alkylene group, and/or Z₃ is absent and Y₃ may be a ketone, a substituted or unsubstituted exocyclic alkylene group, and/or 4 is absent and Y₄ may be a ketone, a substituted or unsubstituted exocyclic alkylene group; and

X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;

or a pharmaceutically acceptable salt thereof.

In a further embodiment, the epidithiodioxopiprazine is represented by Formula V:

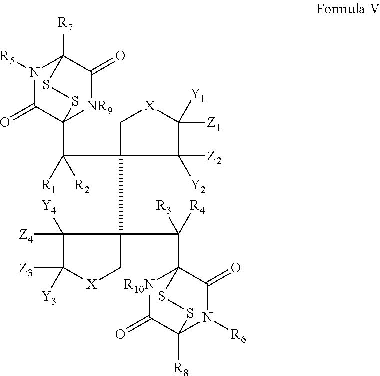

wherein

R₁-R₄ taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or

R₁ is absent and R₂ may be a ketone, a substituted or unsubstituted exocyclic alkylene group, and/or R₃ is absent and R₄ may be a ketone, a substituted or unsubstituted exocyclic alkylene group;

R₅-R₁₀, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;

Y₁-Y₄ and Z₁-Z₄, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or

Y₁ and Y₂, Z₁ and Z₂, Y₃ and Y₄, and/or Z₃ and Z₄, taken together with the atoms to which they are attached, may be C₃-C₂₀ cyclic, substituted C₃-C₂₀ cyclic, heterocyclic, or substituted heterocyclic group; or

Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; or

Z₃ and Z₄ are absent and Y₃ and Y₄, taken together with the atoms to which they are attached may be a π-bond, phenyl, substituted phenyl, aryl, substituted aryl, heteroaryl, substituted heteroaryl, polyaryl, or substituted polyaryl; or

Z₁ is absent and Y₁ may be a ketone, a substituted or unsubstituted exocyclic alkylene group, and/or Z₂ is absent and Y₂ may be a ketone, a substituted or unsubstituted exocyclic alkylene group, and/or Z₃ is absent and Y₃ may be a ketone, a substituted or unsubstituted exocyclic alkylene group, and/or Z₄ is absent and Y₄ may be a ketone, a substituted or unsubstituted exocyclic alkylene group; and

X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;

or a pharmaceutically acceptable salt thereof.

In one embodiment, the epidithiodioxopiprazine is Verticillin A, as shown in the following structure:

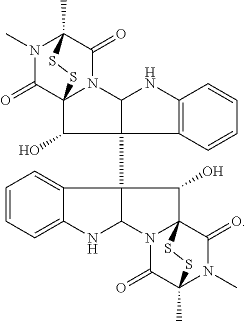

In another embodiment, the epidithiodioxopiprazine is 11-deoxyverticillin, as shown in the following structure:

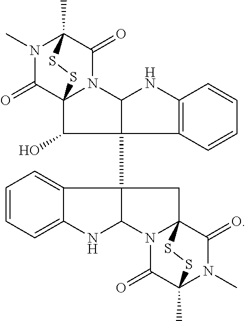

In another embodiment, the epidithiodioxopiprazine is 11,11′-dideoxyverticillin, as shown in the following structure:

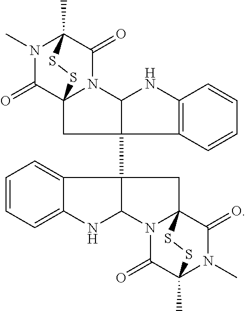

In another embodiment, the epidithiodioxopiprazine is Verticillin 13, as shown in the following structure:

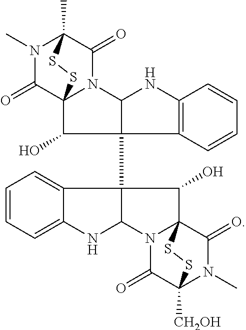

In another embodiment, the epidithiodioxopiprazine is Verticillin D, as shown in the following structure:

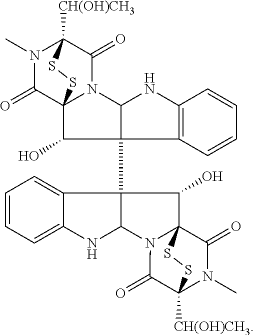

In another embodiment, the epidithiodioxopiprazine is Verticillin E, as shown in the following structure:

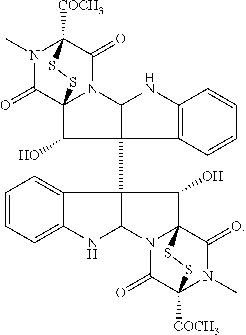

In another embodiment, the epidithiodioxopiprazine is Verticillin F, as shown in the following structure:

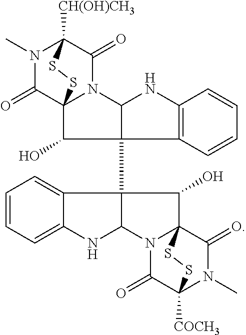

In another embodiment, the epidithiodioxopiprazine is Chaetocin, as shown in the following structure:

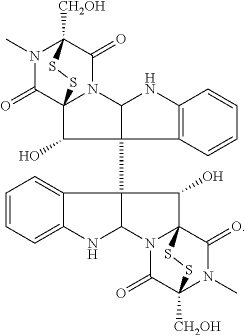

In one embodiment, the epidithiodioxopiprazine is Gliotoxin, as shown in the following structure:

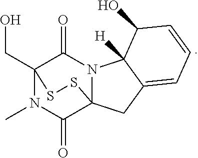

In one embodiment, the epidithiodioxopiprazine is Chaetomin, as shown in the following structure:

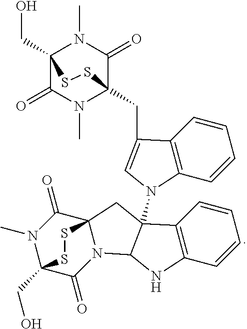

The compounds described herein may have one or more chiral centers and thus exist as one or more stereoisomers. Such stereoisomers can exist as a single enantiomer, a mixture of diastereomers or a racemic mixture.

As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

2. Isolation and/or Synthesis of Epidithiodioxopiprazines

The epidithiodioxopiprazines useful in the compositions and methods described herein can be isolated from natural sources or synthesized de novo. A variety of epidithiodioxopiprazines have been described, for example, by Bible, et al. (U.S. Patent Application Publication No. 2009/0264421), which is incorporated herein by reference in its entirety.

Epidithiodioxopiprazines are a well-known class of natural products. A variety of epidithiodioxopiprazines can be isolated from natural resources, particularly mycoparasites and other fungi. For example, Verticillin A can be isolated from the imperfect fungus Verticillium sp. strain TM-759. It has been shown to possess antimicrobial, anti-viral, and anti-tumor properties (Katagiri K, et al. J Antibiot (Tokyo). 1970 August; 23(8):420-2). Verticillia A can also be isolated from the fresh fruiting bodies of Verticillium sp-infected Amanita flavorubescens Alk collected from Yunnan Province, China. Derivatives of Verticillin A have been isolated from the mycelium of a marine-derived fungus of the genus Penicillium (see Son, et al. Nat. Prod. Lett 1999, 13(3):213-22). Similarly, other epidithiodioxopiprazines, including Verticillins D, E, and F can be isolated from Gliocladium catenulatum (see Joshi, et al. J Nat. Prod 1999, 62(5):730-3). Derivatives of these natural products can be readily prepared using standard techniques well documented in synthetic organic chemistry (see, for example, March, “Advanced Organic Chemistry,” 4^{th} Edition, 1992, Wiley-Interscience Publication, New York).

Epidithiodioxopiprazines can also be synthesized de novo. For example, the total synthesis of (+)-11,11′-dideoxyverticillin A has recently been reported (Kim, et al. Science 2009, 324(5924): 238-41).

3. Concentrations of Epidithiodioxopiprazines

Compositions containing one or more epidithiodioxopiprazines are disclosed. The compositions preferably contain an amount of one or more epidithiodioxopiprazines effective to sensitize a TRAIL-resistant cancer cell to TRAIL-induced apoptosis. For example, it has been shown that as low as 10 nM Verticillin A can effectively overcome TRAIL resistance of human cancer cells. Therefore, in some embodiments, the composition can contain about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, or 1000 nM of one or more epidithiodioxopiprazines.

In some embodiments, the epidithiodioxopiprazine is Verticillin A. The compositions can contain an amount of Verticillin A effective to inhibit growth of cancer cells, such as hepatocarcinoma cells, e.g., HepG2 cells. It has been shown that Verticillin A has an IC₅₀ of less than 200 nM for multiple types of cancer cells. Therefore, in some embodiments, the composition can contain about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, or 1000 nM Verticillin A.

In further embodiments, the epidithiodioxopiprazine is Verticillin B, D, E, or F, 11-deoxyverticillin, 11,11′-dideoxyverticillin, Chaetocin, Gliotoxin, or Chaetomin, or combinations thereof. In these cases, the composition can contain about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, or 1000 nM epidithiodioxopiprazine.

B. Death Receptor Agonists

Epidithiodioxopiprazines sensitize cells to death receptor-induced apoptosis. Therefore, the disclosed compositions containing one or more epidithiodioxopiprazines can be co-administered with death receptor agonists. In some embodiments, a composition containing Verticillin A is co-administered with a death receptor agonist. In further embodiments, a composition containing Verticillin B, D, E, or F, 11-deoxyverticillin, 11,11′-dideoxyverticillin, Chaetocin, Gliotoxin, or Chaetomin is co-administered with a death receptor agonist.

In some embodiments, the composition containing one or more epidithiodioxopiprazines is provided in a kit containing a composition containing a death receptor agonist. In one embodiment, the composition containing Verticillin A is provided in a kit containing a composition containing a death receptor agonist. In further embodiments, the composition containing Verticillin B, D, E, or F, 11-deoxyverticillin, 11,11′-dideoxyverticillin, Chaetocin, Gliotoxin, or Chaetomin is provided in a kit containing a composition containing a death receptor agonist.

In some embodiments, the composition containing one or more epidithiodioxopiprazines further contains a death receptor agonist. In one embodiment, the composition containing Verticillin A further contains a death receptor agonist. In further embodiments, the composition containing Verticillin B, D, E, or F, 11-deoxyverticillin, 11,11′-dideoxyverticillin, Chaetocin, Gliotoxin, or Chaetomin further contains a death receptor agonist.

In some embodiments, compositions containing one or more epidithiodioxopiprazines described by Formulas I-V and further containing one or more death receptor agonists increases cell death by more than 10%, 15%, 20%, 25%, 30%, 35%, 40%, 45%, 50%, 55%, 60%, 65%, 70%, 75%, 80%, 90%, or 100% as compared to administration of the death receptor agonist alone. In one embodiment, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin.

Death receptors include, for example, TNFR1, Fas, DR3, DR4, DR5, DR6 and LTβR. Preferably, the death receptor is DR4 or DR5. Suitable death receptor agonists include any substance (molecule, drug, protein, etc.) that is capable of binding a death receptor on a cell and initiating apoptosis. The death receptor agonist can be a natural ligand of a death receptor, including fragments or variants of the natural ligand. The death receptor agonist can be an antibody that binds and activates a death receptor. The death receptor agonist can be a compound, such as a small molecule identified from a compound library.

1. Death Receptor Ligands

The death receptor agonist can be a death receptor ligand that initiates apoptosis when it binds a death receptor on a cell. For example, death receptor ligand can be a member of the TNF superfamily. In preferred embodiments, the death receptor ligand is TNF-related apoptosis-inducing ligand (TRAIL). In other embodiments, the death receptor ligand is Fas ligand.

TRAIL has a strong apoptosis-inducing activity against cancer cells. Unlike other death-inducing ligands of the TNF superfamily such as TNFα and Fas ligand, TRAIL preferentially induces apoptosis of tumor cells, having little or no effect on normal cells. At least five receptors for TRAIL have been identified, two of which, DR4 (TRAIL-R1) and DR5 (TRAIL-R2), are capable of transducing the apoptosis signal whereas the other three (TRAIL-R3, TRAIL-R4 and OPG) serve as decoy receptors to block TRAIL-mediated apoptosis. The intracellular segments of both DR4 and DR5 contain a death domain and transduce an apoptosis signal through a FADD- and caspase 8-dependent pathway. Administration of the recombinant soluble form of TRAIL induces significant tumor regression without systemic toxicity in animal models. In humans, however, TRAIL has been shown to elicit side effects such as liver toxicity. Therefore, alternative agonists of TRAIL receptors have been developed.

2. Death Receptor-Specific Antibodies

The death receptor agonist can be an apoptosis-inducing antibody that binds the death receptor. For example, the death receptor agonist can be an antibody specific for a death receptor, such that the antibody activates the death receptor. The agonist can be an antibody specific for DR4 or DR5. For example, the agonist can be a DR5 antibody having the same epitope specificity, or secreted by, a mouse-mouse hybridoma having ATCC Accession Number PTA-1428 (e.g., the TRA-8 antibody), ATCC Accession Number PTA-1741 (e.g., the TRA-1 antibody), ATCC Accession Number PTA-1742 (e.g., the TRA-10 antibody), or ATCC Accession Number PTA-3798 (e.g., the 2E12 antibody). The death receptor agonist can be death receptor LTβR mAb (e.g., Biolegend Inc. clone 31G4D8).

The term “antibodies” refers to polyclonal, monoclonal, or recombinant antibodies. In addition to intact immunoglobulin molecules, also included in the term “antibodies” are fragments, polymers, complexes, or multimers (e.g., diabodies, triabodies, and tetrabodies) of those immunoglobulin molecules, and human or humanized versions of immunoglobulin molecules or fragments thereof, as long as they are chosen for their ability to activate a death receptor, such as DR4 or DR5. The term “antibody” encompasses chimeric antibodies and hybrid antibodies, with single, dual or multiple antigen or epitope specificities. Generally, useful antibody fragments retain at least the Fv region of the immunoglobulin. Antibody fragments include F(ab′)₂, Fab′, and Fab fragments. Also disclosed are derivatives, combinations, modifications, homologs, mimetics, and conservative variants of the disclosed antibodies that can bind and activate a death receptor, such as DR4 or DR5.

C. Anti-Neoplastic Agents

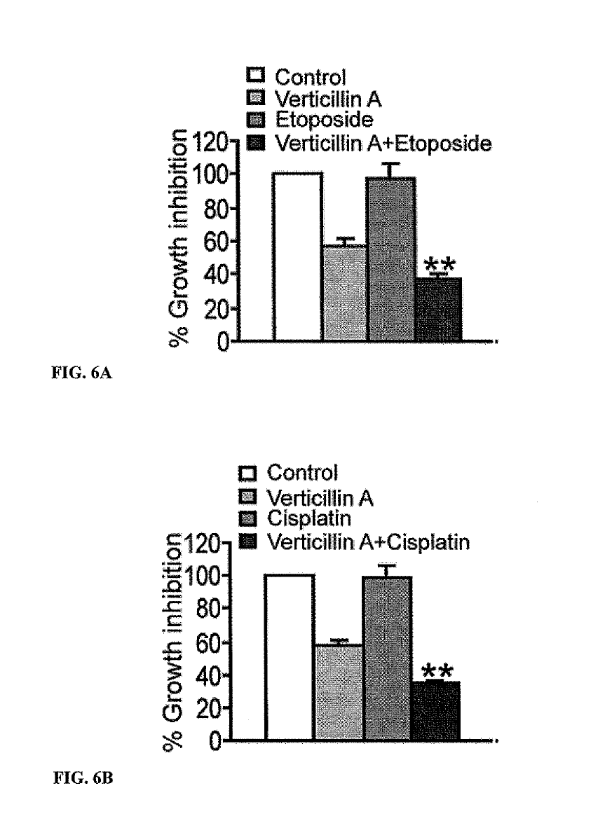

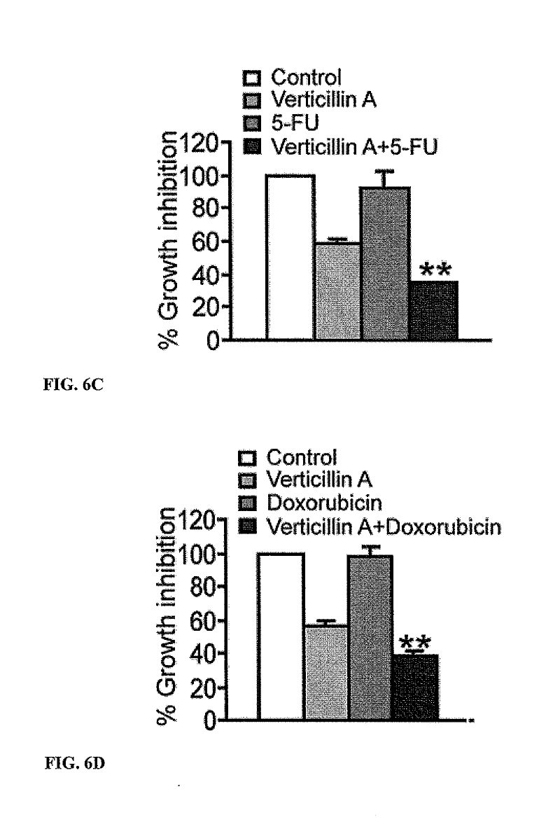

Epidithiodioxopiprazines can increase the efficacy of anti-neoplastic agents. For example, in Example 5, Verticillin A is shown to increase the efficacy of etoposide, cisplatin, 5-FU, and doxorubicin. Specifically, SW620 cells pretreated with Verticillin. A, were treated with Etoposide (1 μg/ml), Cisplatin (1 μg/ml), 5-FU (0.1 μg/ml) and Doxorubicin (0.01 μg/ml), respectively. At lower doses, etoposide, cisplatin, 5-FU, and doxorubicin alone exerted minimal inhibitory effects. However, combination of these drugs with Verticillin A significantly increased the tumor growth inhibitory effects of these drugs (FIG. 6). Therefore, Verticillin A can be used to reduce the effective dose of these drugs to reduce the toxicity of existing anticancer drugs.

Therefore, the disclosed compositions containing one or more epidithiodioxopiprazines can further contain one or more anti-neoplastic agents including, but not limited to alkylating agents (such as cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil (5-FU), gemcitabine, methotrexate, cytosine arabinoside, fludarabine, and floxuridine), antimitotics (including taxanes such as paclitaxel and decetaxel and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as irinotecan and topotecan and derivatives of epipodophyllotoxins such as amsacrine, etoposide, etoposide phosphate, and teniposide). In one embodiment, a composition containing Verticillin A further contains etoposide, cisplatin, 5-FU, doxorubicin, or a combination thereof. In further embodiments, a composition containing Verticillin B, D, E, or F, 11-deoxyverticillin, 1′,11′-dideoxyverticillin, Chaetocin, Gliotoxin, or Chaetomin further contains etoposide, cisplatin, 5-FU, doxorubicin, or a combination thereof.

In some embodiments, compositions containing one or more epidithiodioxopiprazines and further containing one or more classes of other anti-cancer (anti-neoplastic) agents increase cell death by more than 10%, 15%, 20%, 25%, 30%, 35%, 40%, 45%, 50%, 55%, 60%, 65%, 70%, 75%, 80%, 90%, or 100% as compared to administration of the anti-cancer agent alone. In some embodiments, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin.

The compositions containing one or more epidithiodioxopiprazines can further contain one or more additional radiosensitizers, such as gemcitabine, pentoxifylline, or vinorelbine. In one embodiment, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin.

D. Additional Therapeutics

In other embodiments, the disclosed compositions can further contain one or more additional active agents (e.g., therapeutics agents).

The composition can further contain one or more of classes of antibiotics, such as aminoglycosides, cephalosporins, chloramphenicol, clindamycin, erythromycins, fluoroquinolones, macrolides, azolides, metronidazole, penicillins, tetracyclines, trimethoprim-sulfamethoxazole, or vancomycin.

The composition can contain one or more classes of steroids, such as andranes (e.g., testosterone), cholestanes (e.g., cholesterol), cholic acids (e.g., cholic acid), corticosteroids (such as dexamethasone and prednisone), estraenes (e.g., estradiol), or pregnanes (e.g., progesterone).

The composition can contain one or more classes of narcotic and non-narcotic analgesics, such as morphine, codeine, heroin, hydromorphone, levorphanol, meperidine, methadone, oxydone, propoxyphene, fentanyl, naloxone, buprenorphine, butorphanol, nalbuphine, or pentazocine.

The composition can contain one or more classes of anti-inflammatory agents, including, but not limited to salicylates (such as acetylsalicylic acid, diflunisal and salsalate), propionic acid derivatives (such as ibuprofen, naproxen, fenoprofen, ketoprofen, flurbiprofen, oxaprozin, andioxoprofen), acetic acid derivatives (such as indomethacin, sulindac, etodolac, and ketorolac), enolic acid (oxicam) derivatives (such as piroxicam, meloxicam, tenoxicam, droxicam, lornoxicam, and isoxicam), fenamic acid derivatives (such as mefenamic acid, meclofenamic acid, flufenamic acid, and tolfenamic acid), selective COX-2 inhibitors, sulphonanilides (such as nimesulide), and COX/LOX inhibitors (such as licofelone)

The composition can contain one or more classes of anti-histaminic agents, such as ethanolamines (e.g., diphenhydramine carbinoxamine), ethylenediamines (e.g., tripelennamine pyrilamine), alkylamines (e.g., chlorpheniramine, dexchlorpheniramine, brompheniramine, triprolidine), or other anti-histamines such as astemizole, loratadine, fexofenadine, bropheniramine, clemastine, acetaminophen, pseudoephedrine, and triprolidine.

E. Pharmaceutical Compositions

A pharmaceutical composition containing therapeutically effective amounts of one or more epidithiodioxopiprazines and a pharmaceutically acceptable carrier is disclosed. In some embodiments, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin. Pharmaceutical carriers suitable for administration of the disclosed compounds include any such carriers known to those skilled in the art to be suitable for the particular mode of administration.

The disclosed compositions can be formulated into suitable pharmaceutical preparations such as solutions, suspensions, tablets, dispersible tablets, pills, capsules, powders, delayed and/or sustained release formulations, or elixirs for oral administration, or in sterile solutions or suspensions for parenteral administration. In one embodiment, one or more epidithiodioxopiprazines are formulated into pharmaceutical compositions using techniques and procedures well known in the art. In some embodiments, Verticillin A is formulated into pharmaceutical compositions. In further embodiments, Verticillin B, D, E, or F, 11-deoxyverticillin, 11,11′-dideoxyverticillin, Chaetocin, Gliotoxin, or Chaetomin are formulated into pharmaceutical compositions.

In some embodiments, the disclosed compositions are formulated for single dosage administration. To formulate a composition, the weight fraction of active agent(s) is dissolved, suspended, dispersed or otherwise mixed in a selected carrier at an effective concentration such that the treated condition is relieved or one or more symptoms are ameliorated. The disclosed active agent(s) can be included in the pharmaceutically acceptable carrier in an amount sufficient to exert a therapeutically useful effect in the absence of undesirable side effects on the patient treated. The therapeutically effective concentration is determined empirically by testing the compounds in in vitro, ex vivo and in vivo systems, and then extrapolated therefrom for dosages for humans. The concentration of active agent(s) in the pharmaceutical composition will depend on absorption, inactivation and excretion rates of the active compound, the physicochemical characteristics of the agent, the dosage schedule, and amount administered as well as other factors known to those of skill in the art.

Dosage forms or compositions containing active agent(s) in the range of 0.005% to 100% with the balance made up from non-toxic carrier may be prepared. Methods for preparation of these compositions are known to those skilled in the art. The contemplated compositions may contain 0.001%-100% active ingredient, or in one embodiment 0.1-95%.

Methods for solubilizing active agents or improving bioavailability may be used. Such methods are known to those of skill in this art, and include, but are not limited to, using cosolvents, such as dimethylsulfoxide (DMSO), using surfactants, such as TWEEN®, or dissolution in aqueous sodium bicarbonate. The pharmaceutical compositions of one or more of the active agents can be incorporated into a polymer matrix, for example, hydroxypropylmethyl cellulose, gel, permeable membrane, osmotic system, multilayer coating, microparticle, nanoparticle, liposome, microsphere, nanosphere, or the like. The active agent(s) may be suspended in micronized or other suitable form or may be derivatized (e.g., by adding one or more polyethylene glycol chains) to produce a more soluble active product or improve bioavailability. To optimize absorption, distribution, metabolism, and excretion, or improve oral bioavailability, the active agent(s) may be provided as prodrugs (i.e. in an inactive or significantly less active form which is metabolised in vivo into an active agent). The active agent(s) described herein may also be conjugated to a biomolecule, including, but not limited to a protein or nucleic acid, to affect bioavailability.

Liquid pharmaceutically administrable compositions can, for example, be prepared by dissolving, dispersing, or otherwise mixing the active agent(s) and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

1. Compositions for Oral Administration

Oral pharmaceutical dosage forms can be either solid, gel, or liquid. The solid dosage forms can be tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating.

The active agent(s), or a pharmaceutically acceptable salt(s) thereof, can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes, and cellulose acetate phthalate.

2. Injectables, Solutions, and Emulsions

The pharmaceutical composition can be in a parenteral administration form. Injectables can be prepared in conventional forms, either as liquid solutions or suspensions, solid forms suitable for solution or suspension in liquid prior to injection, or as emulsions. The injectables, solutions, and emulsions may also contain one or more excipients. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. The percentage of active compound contained in such parenteral compositions is highly dependent on the specific nature thereof, as well as the activity of the compound and the needs of the subject.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The unit-dose parenteral preparations can be packaged in an ampoule, a vial or a syringe with a needle. All preparations for parenteral administration should be sterile, as is known and practiced in the art. The injectable compositions described herein can be optimized for local and/or systemic administration.

The active agent(s) may be suspended in micronized or other suitable form. The active agent(s) may also be derivatized to produce a more soluble active product or to produce a prodrug. The form of the resulting mixture depends upon a number of factors, including the intended mode of administration and the solubility of the active agent(s) in the selected carrier or vehicle. The effective concentration is sufficient for ameliorating the symptoms of the condition and may be empirically determined.

Implantation of a slow-release or sustained-release system, such that a constant level of dosage is maintained is also contemplated herein. In such cases, the active agent(s) provided herein can be dispersed in a solid matrix optionally coated with an outer rate-controlling membrane. The compound diffuses from the solid matrix (and optionally through the outer membrane) sustained, rate-controlled release. The solid matrix and membrane may be formed from any suitable material known in the art including, but not limited to, polymers, bioerodible polymers, and hydro gels.

3. Lyophilized Powders

Lyophilized powders can be reconstituted for administration as solutions, emulsions and other mixtures. They may also be reconstituted and formulated as solids or gels. The sterile, lyophilized powder can be prepared by dissolving a disclosed active agent, such as Verticillin A, or a pharmaceutically acceptable salt thereof, in a suitable solvent. The solvent may contain an excipient which improves the stability or other pharmacological component of the powder or reconstituted solution, prepared from the powder. The solvent may also contain a buffer, such as citrate, sodium or potassium phosphate or other such buffer known to those of skill in the art at. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

4. Targeted Formulations

The disclosed active agent(s), or pharmaceutically acceptable salts thereof, can be formulated to be targeted to a particular tissue, receptor, or other area of the body of the subject to be treated. Many such targeting methods are well known to those of skill in the art. In one embodiment, liposomal suspensions, including tissue-targeted liposomes, such as tumor-targeted liposomes, may also be suitable as pharmaceutically acceptable carriers. These may be prepared according to methods known to those skilled in the art.

5. Delayed and Sustained Release Formulations

Compositions can be formulated to provide immediate or delayed release of one or more of the active agent(s), including epidithiodioxopiprazines. Also disclosed are sustained release formulations to maintain therapeutically effective amounts of one or more active agents, including epidithiodioxopiprazines, over a period of time. In some embodiments, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin.

In compositions containing multiple active agents, the active agents may be individually formulated to control the duration and/or time release of each active agent. In one embodiment, a composition containing one or more epidithiodioxopiprazines further contains a death receptor agonist formulated for sustained and/or timed release. In one embodiment, a composition containing one or more epidithiodioxopiprazines further contains an anti-neoplastic agent formulated for sustained and/or timed release.

Such sustained and/or timed release formulations may be made by sustained release means of delivery devices that are well known to those of ordinary skill in the art. These pharmaceutical compositions can be used to provide slow or sustained release of one or more of the active agents using, for example, hydroxypropylmethyl cellulose, other polymer matrices, gels, permeable membranes, osmotic systems, multilayer coatings, microparticles, nanoparticles, liposomes, microspheres, nanospheres or the like. The active agents may also be suspended, micronized, or derivatized to vary release of the active ingredient(s).

### III. Methods

A. Sensitizing cells to death receptor agonists

A method of selectively inducing apoptosis in a target cell expressing a death receptor is provided. The method generally involves contacting the cell with a composition containing an effective amount of one or more epidithiodioxopiprazines and a death receptor agonist that binds the death receptor on the target cell. In some embodiments, the method involves contacting the cell with Verticillin A and a death receptor agonist that binds the death receptor on the target cell. In further embodiments, the method involves contacting the cell with Verticillin B, D, E, or F, 11-deoxyverticillin, 11,11′-dideoxyverticillin, Chaetocin, Gliotoxin, or Chaetomin, and a death receptor agonist that binds the death receptor on the target cell. In all such embodiments, the epidithiodioxopiprazine and the death receptor agonist can be in the same composition or in separate compositions.

It has been shown that as low as 10 nM Verticillin A can effectively overcome TRAIL resistance of human cancer cells. In some embodiments, the method involves contacting the cell with one or more epidithiodioxopiprazines at a concentration of about 10 to 200 nM epidithiodioxopiprazine, including about 10 to 100 nM, 20 to 100 nM, 10 to 50 nM epidithiodioxopiprazine. In some methods, cells are contacted with one or more epidithiodioxopiprazines at a concentration of about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, or 1000 nM epidithiodioxopiprazine.

Epidithiodioxopiprazines sensitize the cell to death receptor-induced apopotis. In some embodiments, compositions containing epidithiodioxopiprazines and further containing one or more death receptor agonists increases cell death by more than 10%, 15%, 20%, 25%, 30%, 35%, 40%, 45%, 50%, 55%, 60%, 65%, 70%, 75%, 80%, 90%, or 100% as compared to administration of the death receptor agonist alone. In preferred embodiments, the cell is resistant to apoptosis induced by the death receptor agonist. Therefore, the method can in some embodiments induce apoptosis in a target cell that will not undergo apoptosis when contacted with the same amount of death receptor agonist alone. In other embodiments, the method reduces the amount of death receptor agonist required to induce apoptosis in the target cell.

B. Inhibiting DNA Methylation

DNA methylation involves the addition of a methyl group to DNA. For example, when a methyl group is added to the number 5 carbon of the cytosine pyrimidine ring, gene expression is reduced. In adult somatic tissues, DNA methylation typically occurs in a CpG dinucleotide context; non-CpG methylation is prevalent in embryonic stem cells. CpG sites are regions of DNA where a cytosine nucleotide occurs next to a guanine nucleotide in the linear sequence of bases along its length. The term “CpG” refers to a cytosine and guanine separated by a phosphate, which links the two nucleosides together in DNA.

Unmethylated CpGs are often grouped in clusters called CpG islands, which are present in the 5′ regulatory regions of many genes. In many disease processes, such as cancer, gene promoter CpG islands acquire abnormal hypermethylation, which results in transcriptional silencing that can be inherited by daughter cells following cell division. Alterations of DNA methylation have been recognized as an important component of cancer development. Hypomethylation, in general, arises earlier and is linked to chromosomal instability and loss of imprinting, whereas hypermethylation is associated with promoters and can arise secondary to gene (oncogene suppressor) silencing, but might be a target for epigenetic therapy.

A method of inhibiting DNA methylation in a cell is provided. The method can involve contacting the cell with a composition containing one or more epidithiodioxopiprazines described by Formulas I-V. In some embodiments, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin.

The method of inhibiting DNA methylation can be due to direct or indirect inhibition. In one embodiment, indirect inhibition of DNA methylation can occur by one of the disclosed compositions acting on another molecule wherein that molecule then inhibits DNA methylation. For example, Verticillin A can activate TET1 which is known to convert 5-methylcytosine to 5-hydroxyl methylcytosine, and eventually to unmodified cytosine to reverse DNA methylation. Therefore, Verticillin A indirectly inhibits DNA methylation.

In preferred embodiments, the method involves inhibiting DNA methylation of CpG islands in the promoter region of one or more tumor suppressor genes, cell cycle related genes, DNA mismatch repair genes, hormone receptors and tissue, cell adhesion molecules, or a combination thereof. For example, the method can involve inhibiting DNA methylation of CpG islands in the promoter region of BNIP3, Neurog1, p15ink4b, RUNX3.

In some embodiments, the method can decrease DNA methylation of CpG islands in the cell by at least 10%, 15%, 20%, 25%, 30%, 35%, 40%, 45%, 50%, 55%, 60%, 65%, 70%, 75%, 80%, 90%, or 100% as compared to a control.

In some embodiments, the method can treat or prevent DNA hypermethylation of CpG islands in the cell. The term “hypermethylation” refers to abnormal methylation of CpG islands that results in transcriptional silencing of a gene. Therefore, in some embodiments, the method can promote transcription of a silenced gene by at least 10%, 15%, 20%, 25%, 30%, 35%, 40%, 45%, 50%, 55%, 60%, 65%, 70%, 75%, 80%, 90%, or 100% as compared to a control.

C. Treating Diseases Associated with Aberrant Cell Survival or Proliferation

Methods and compositions are provided for use in the treatment of diseases associated with inappropriate survival or proliferation of target cells, including those attributable to dysregulation of the apoptosis systems in cancer or in inflammatory and autoimmune diseases.

Inflammatory and autoimmune diseases illustratively include systemic lupus erythematosus, Hashimoto's disease, rheumatoid arthritis, graft-versus-host disease, Sjogren's syndrome, pernicious anemia, Addison disease, scleroderma, Goodpasture's syndrome, Crohn's disease, autoimmune hemolytic anemia, sterility, myasthenia gravis, multiple sclerosis, Basedow's disease, thrombopenia purpura, insulin-dependent diabetes mellitus, allergy, asthma, atopic disease, arteriosclerosis, myocarditis, cardiomyopathy, glomerular nephritis, hypoplastic anemia, rejection after organ transplantation.

Cancers which can be treated using the composition and methods describe herein include sarcomas, lymphomas, leukemias, carcinomas, blastomas, and germ cell tumors. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The provided compositions and methods can further be used to target and selectively induce apoptosis in activated immune cells including activated lymphocytes, lymphoid cells, myeloid cells, and rheumatoid synovial cells (including inflammatory synoviocytes, macrophage-like synoviocytes, fibroblast-like synoviocytes) and in virally infected cells (including those infected with HIV, for example) so long as those targeted cells express or can be made to express the specific death receptors (i.e., DR4 or DR5).

1. Epidithiodioxopiprazines as the Active Ingredient

A method for treating a subject with cancer is also provided. This method involves administering to the subject a composition containing one or more epidithiodioxopiprazines. In some embodiments, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin.

It has been shown, for example, that Verticillin A has an IC₅₀ of less than 200 nM for multiple types of cancer cells. Therefore, in some embodiments, the composition can contain about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 450, 500, 550, 600, 650, 700, 750, 800, 850, 900, 950, or 1000 nM of one or more epidithiodioxopiprazines described by Formulas I-V. In some embodiments, the epidithiodioxopiprazine is Verticillin A. In further embodiments, the epidithiodioxopiprazine is Verticillin B, D, E, or F, 1′-deoxyverticillin, 11,11′-dideoxyverticillin, Chaetocin, Gliotoxin, or Chaetomin.

2. Co-Administration of Epidithiodioxopiprazines with a Death Receptor Agonist

In preferred embodiments, a therapeutic amount of composition containing one or more epidithiodioxopiprazines is co-administered with a therapeutic amount of a composition containing death receptor agonist, wherein the epidithiodioxopiprazine(s) reduce resistance of the cancer cells to the death receptor agonist. In some embodiments, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin.

Epidithiodioxopiprazines enhance the efficacy of TRAIL in suppressing human cancer growth in nude mice at doses as low as 0.125 mg/kg. For example, Verticillin A can enhance the efficacy of TRAIL in suppressing human cancer growth in nude mice at doses as low as 0.125 mg/kg. Therefore, a therapeutic amount of composition containing one or more epidithiodioxopiprazines can contain at least about 0.125 mg/kg of one or more epidithiodioxopiprazines. The therapeutic amount of composition containing one or more epidithiodioxopiprazines can contain about 0.1 mg/kg to about 10 mg/kg, including about 0.125 mg/kg to about 0.5 mg/kg, and about 0.1 to about 1 mg/kg of one or more epidithiodioxopiprazines. The therapeutic dose can be about 0.1 to about 100 mg/m2, including 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, and 100 mg/m2. In one embodiment, the epidithiodioxopiprazine is Verticillin A. In further embodiments, the epidithiodioxopiprazine is Verticillin B, D, E, or F, 11-deoxyverticillin, 11,11′-dideoxyverticillin, Chaetocin, Gliotoxin, or Chaetomin.

The co-administration of epidithiodioxopiprazine and death receptor agonist can be simultaneous or sequential. Simultaneous administration includes the use of a single composition containing both epidithiodioxopiprazines and a death receptor agonist. Simultaneous administration also includes administration of separate compositions of one or more epidithiodioxopiprazines and a death receptor agonist at substantially the same time. “Substantially the same time” includes administration of the second composition within 1 minute of the first composition.

In some embodiments, the compositions are administered sequentially. In this method, the composition containing one or more epidithiodioxopiprazines is administered first to sensitize the target cells prior to administration of the second composition containing the death receptor agonist. For example, the composition containing one or more epidithiodioxopiprazines can be administered from 1 minute to 7 days before administration of the composition containing the death receptor agonist. For example, the composition containing one or more epidithiodioxopiprazines can be administered at least about 1, 2, 3, 4, 5, 6, 7, 8, 9, 10, 11, 12, 13, 14, 15, 16, 17, 18, 19, 20, 25, 30, 35, 40, 45, 50, 60 minutes before administration of the composition containing the death receptor agonist. For example, the composition containing one or more epidithiodioxopiprazines can be administered at least about 1, 2, 3, 4, 5, 6, 7, 8, 9, 10, 11, 12, 13, 14, 15, 16, 17, 18, 19, 20, 21, 22, 23, 24 hours before administration of the composition containing the death receptor agonist. For example, the composition containing one or more epidithiodioxopiprazines can be administered at least about 1, 2, 3, 4, 5, 6, 7 days before administration of the composition containing the death receptor agonist.

Sequential administration can also be accomplished by administering a composition containing one or more epidithiodioxopiprazines and a delayed release formulation of the death receptor agonist.

In one method, a composition containing one or more epidithiodioxopiprazines is administered locally to sensitize the target cells while a composition containing a death receptor agonist is administered systemically. In one method, a composition containing one or more epidithiodioxopiprazines is administered systemically to sensitize the target cells while a composition containing a death receptor agonist is administered locally.

The cancer of the disclosed methods can be any cell in a subject undergoing unregulated growth, invasion, or metastasis. In some aspects, the cancer can be resistant to TRAIL-induced apoptosis.

3. Co-Administration of Epidithiodioxopiprazines with an Anti-Neoplastic Agent

The Examples demonstrate that epidithiodioxopiprazines decreases cancer resistance to anti-neoplastic agents, such as etoposide, cisplatin, 5-FU, and doxorubicin. In Example 5, Verticillin A is shown to increase the efficacy of etoposide, cisplatin, 5-FU, and doxorubicin. Specifically, SW620 cells pretreated with Verticillin A, were treated with Etoposide (1 μg/ml), Cisplatin (1 μg/ml), 5-FU (0.1 μg/ml) and Doxorubicin (0.01 μg/ml), respectively. At lower doses, etoposide, cisplatin, 5-FU, and doxorubicin alone exerted minimal inhibitory effects. However, combination of these drugs with Verticillin A significantly increased the tumor growth inhibitory effects of these drugs (FIG. 6).

Therefore, methods are provided for treating cancer in a subject involving co-administering a composition containing one or more epidithiodioxopiprazines with a composition containing one or more anti-neoplastic agents. The anti-neoplastic agents can be present in the composition at concentrations lower than would be effective if administered without the epidithiodioxopiprazine(s). Therefore, also provided are methods of lowering the effective dose of an anti-neoplastic agent and reducing toxicity of the agent involving co-administering the agent with an effective amount of one or more epidithiodioxopiprazines. Moreover, in some embodiments, co-administration with one or more epidithiodioxopiprazines overcomes tumor cell resistance to the anti-neoplastic agent.

In preferred embodiments, the methods can involve co-administering a composition containing one or more epidithiodioxopiprazines with a composition containing etoposide, cisplatin, 5-FU, doxorubicin, or a combination thereof. In combination with one or more epidithiodioxopiprazines, these drugs can be used at a lower dose to reduce their toxicity while maintaining efficacy. Therefore, the composition can contain an amount of etoposide, cisplatin, 5-FU, or doxorubicin that is lower than the effective amount of these compounds without the epidithiodioxopiprazine(s). In preferred embodiments, the epidithiodioxopiprazine is Verticillin A.

The co-administration of epidithiodioxopiprazine and anti-neoplastic agent can be simultaneous or sequential. Simultaneous administration includes the use of a single composition containing both one or more epidithiodioxopiprazines and one or more anti-neoplastic agents. Simultaneous administration also includes administration of separate compositions of one or more epidithiodioxopiprazines and one or more anti-neoplastic agents at substantially the same time. “Substantially the same time” includes administration of the second composition within 1 minute of the first composition.

In some embodiments, the compositions are administered sequentially. In this method, the composition containing one or more epidithiodioxopiprazines is administered first to sensitize the target cells prior to administration of the second composition containing one or more anti-neoplastic agents. For example, the composition containing one or more epidithiodioxopiprazines can be administered from 1 minute to 7 days before administration of the composition containing one or more anti-neoplastic agents. For example, the composition containing one or more epidithiodioxopiprazines can be administered at least about 1, 2, 3, 4, 5, 6, 7, 8, 9, 10, 11, 12, 13, 14, 15, 16, 17, 18, 19, 20, 25, 30, 35, 40, 45, 50, 60 minutes before administration of the composition containing one or more anti-neoplastic agents. For example, the composition containing one or more epidithiodioxopiprazines can be administered at least about 1, 2, 3, 4, 5, 6, 7, 8, 9, 10, 11, 12, 13, 14, 15, 16, 17, 18, 19, 20, 21, 22, 23, 24 hours before administration of the composition containing one or more anti-neoplastic agents. For example, the composition containing one or more epidithiodioxopiprazines can be administered at least about 1, 2, 3, 4, 5, 6, 7 days before administration of the composition containing the one or more anti-neoplastic agents.

Sequential administration can also be accomplished by administering a composition containing one or more epidithiodioxopiprazines and a delayed release formulation of one or more anti-neoplastic agents.

In one method, a composition containing one or more epidithiodioxopiprazines is administered locally to sensitize the target cells while a composition containing one or more anti-neoplastic agents is administered systemically. In one method, a composition containing one or more epidithiodioxopiprazines is administered systemically to sensitize the target cells while a composition containing one or more anti-neoplastic agents is administered locally.

In some embodiments, the method of co-administering compositions containing epidithiodioxopiprazines with compositions containing one or more classes of other anti-cancer (anti-neoplastic) agents increase cell death by more than 10%, 15%, 20%, 25%, 30%, 35%, 40%, 45%, 50%, 55%, 60%, 65%, 70%, 75%, 80%, 90%, or 100% as compared to administration of the anti-cancer agent alone.

4. Therapeutic Administration

The disclosed compositions, including pharmaceutical compositions, may be administered in a number of ways depending on whether local or systemic treatment is desired, and on the area to be treated. For example, the disclosed compositions can be administered orally, parenterally (e.g., intravenous, intramuscular, intraperitoneal, subcutaneous injection), topically or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The disclosed compositions may be administered prophylactically, e.g., to patients or subjects who are at risk for cancer growth or metastasis. Thus, the method can further comprise identifying a subject at risk for cancer growth or metastasis prior to administration of the disclosed compositions.

The exact amount of the compositions required will vary from subject to subject, depending on the species, age, sex, weight and general condition of the subject, extent of the disease in the subject, route of administration, whether other drugs are included in the regimen, and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. Dosage can vary, and can be administered in one or more dose administrations daily, for one or several days. Guidance can be found in the literature for appropriate dosages for given classes of pharmaceutical products.

### EXAMPLES

### Example 1

### Purification and Identification of Verticillin A as an Anti-Tumor Cytotoxic Agent

Materials and Methods

Purification and Identification of Verticillin A

The fresh fruiting bodies of Verticillium sp-infected Amanita flavorubescens Alk was collected from Yunnan Province, China, and authenticated by Prof. Yongchang Zhao (Yunnan Academy of Agriculture Sciences, Kunming, China, voucher number 20051053). The fresh bodies of the fungus (1500 g) were first lyophilized and then extracted successively by light petroleum and ethyl acetate. The ethyl acetate extract (1.2 g) was fractionated by countercurrent chromatography using a two-phase solvent system composed of light petroleum, chloroform and acetonitrile with a volume ratio of 6:1:3. Fractions were assayed for their cytotoxicity against HepG2 cells in MTT assays. One fraction exhibited significant cytotoxicity and was subjected to semi-preparative chromatography on a reverse-phase C8 column (Hypersil ODS 20×250 mm), eluted by acetonitrile and water with a gradient from 10 to 100%. A cytotoxic compound (10 mg) with a purity of 99.0% was obtained. This compound was further determined to have a molecular formula of C₃₀H₂₈N₆O₆S₄ and molecular weight of 696.3. The structure of this compound was determined by electro-spray ionization mass spectrometry (EST-MS) and one- and two-dimensional nuclear magnetic resonance (NMR) spectra as verticillin A. In all experiments, verticillin A was dissolved in DMSO and diluted to working solution or culture medium or HBSS.

Statistical Analysis

Where indicated, data were represented as the mean±SD. Statistical analysis was carried out using two-sided t test, with p-values<0.05 considered statistically significant.

Results

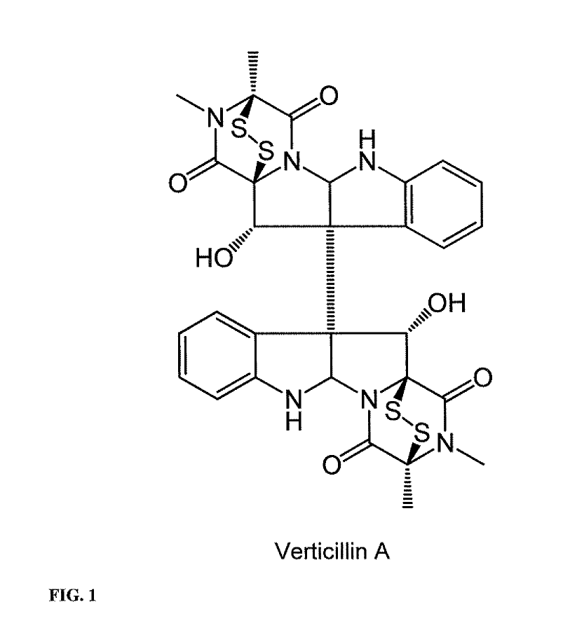

Poison mushrooms have been shown to contain natural anti-tumor substances (Wasser S P. Appl Microbial Biotechnol 2002 60(3):258-74). To purify these natural anti-tumor agents, the fresh bodies of poison mushroom (Amanita flavorubescens Alk) infected by fungus Verticillium sp were extracted, fractionated and screened for anti-tumor cytotoxicity as described in the materials and methods. From approximately 1500 g fresh mushroom, a compound (approximately 10 mg) was purified with 99% purity and potent inhibitory activity against HepG2 cells. This compound has a formula of C₃₀H₂₈N₆O₆S₄ and molecular weight of 696.3. Analysis with electro-spray ionization mass spectrometry and nuclear magnetic resonance (NMR) spectra, in combination with comparing the crystal structure with the database (Minato H, et al. J Chem Soc Perkin 1973 17:1819-25) identified this compound as Verticillin A (FIG. 1).

### Example 2

### Verticillin A Inhibits the Growth of Heptocarcinoma Cells In Vitro

Materials and Methods

Cell Lines

All cell lines used in this study were obtained from American Type Culture Collection (Mannassas, Va.). Cells were maintained in Dulbecco's modified Eagle's medium (DMEM) or Roswell Park Memorial Institute medium (RPMI) (Invitrogen, Carlsbad, Calif.) supplemented with 10% (v/v) fetal bovine serum (FBS), in 37° C. humidified 5% CO₂ incubator.

Mice

Athymic mice were obtained from NCI Frederick mouse facility. Six to eight weeks old female mice were used. Mice were housed in the Medical College of Georgia animal facility. Experiments and care/welfare were in agreement with federal regulations and an approved protocol by the MCG/IACUC committee.

Cell Viability Assays

For cell viability assay, cells were seeded in wells of 96-well plates for 2 days and then treated with different concentrations of mushroom extract fractions or purified verticillin A for 2 days. The cells were then incubated with MTT (3-(4,5-dimethylthiazol-2-yl)-2,5-diphenyltetrazolium bromide) using the MTT assay kit (ATCC, Manassas, Va.) according to the manufacturer's instructions.

DNA Fragmentation Assay

Tumor cells were lysed in lysis buffer (5 mM Tris-HCl, pH 8.0, 100 mM Ethylenediaminetetraacetic acid (EDTA), 1% (w/v) Sodium dodecyl sulfate (SDS), and proteinase K) at 45° C. for 2 h. RNA was removed by incubation with RNase A. Genomic DNA was extracted by phenol/chloroform/isoamyl alcohol (25:24:1) and precipitated with ethanol. The purified genomic DNA was then analyzed by electrophoresis in a 1.2% agarose gel.

Statistical Analysis

Where indicated, data were represented as the mean±SD. Statistical analysis was carried out using two-sided t test, with p-values<0.05 considered statistically significant.

Results

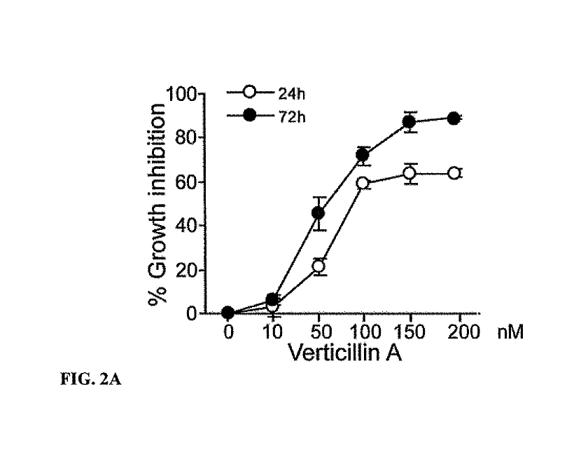

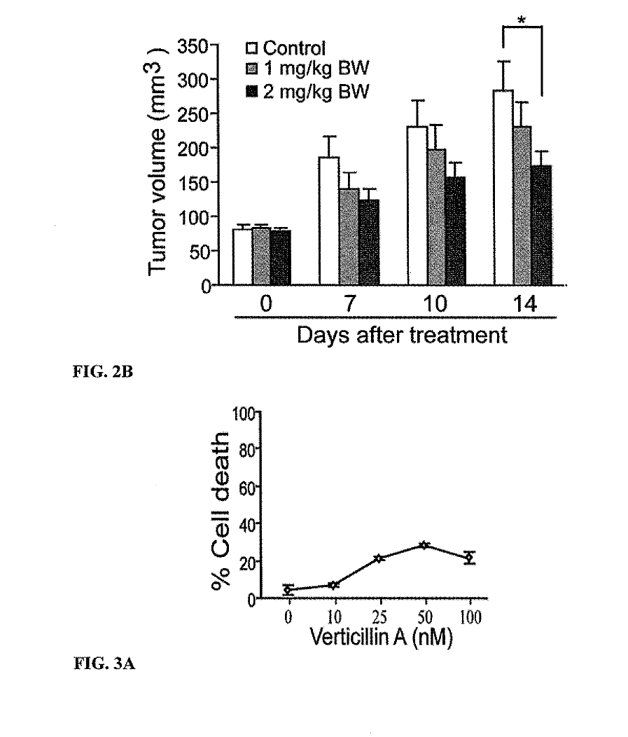

The growth inhibitory effort of the purified verticillin A was examined on HepG2 cells. Tumor cells were cultured in the presence of different concentrations of verticillin A and analyzed for the growth by MTT assay 24 and 72 h later, respectively. As low as 10 nM verticillin A exhibited growth inhibitory effort on HepG2 cells and the inhibitory effect reached plateau at 100-150 nM (FIG. 2A). To determine whether the growth inhibitory effect is due to increased tumor cell death, genomic DNA was isolated from the treated tumor cells and analyzed by agarose gel electrophoresis. It is clear that verticillin A induces genomic DNA fragmentation in the tumor cells. Consistent with the degree of growth inhibition, the level of DNA fragmentation increased with the increase of verticillin A concentration, indicating that verticillin A inhibits tumor cell growth at least partially through inducing HepG2 cell apoptosis.

To determine whether the growth inhibitory effect of verticillin A can be extended to in vivo tumor growth inhibition, HepG2 cells were injected subcutaneously to athymic mice. Tumor-bearing mice were then treated with verticillin A by intravenous injection. Verticillin A exhibited tumor growth inhibitory effects at a dose-dependent manner and significantly inhibited HepG2 tumor growth at a dose of 2 mg/kg body weight (FIG. 2B).

### Example 3

### Verticillin A is a Potent Suppressor of Multiple Types of Tumor Cells

Materials and Methods

Cell Lines

All cell lines used in this study were obtained from American Type Culture Collection (Mannassas, Va.). Cells were maintained in DMEM or RPMI medium (Invitrogen, Carlsbad, Calif.) supplemented with 10% (v/v) fetal bovine serum (FBS), in 37° C. humidified 5% CO₂ incubator.

Statistical Analysis

Where indicated, data were represented as the mean±SD. Statistical analysis was carried out using two-sided t test, with p-values<0.05 considered statistically significant.

Results

To determine whether verticillin A inhibits other types of tumor cell growth, Mammary carcinoma (Bcap-37 and MCF-7), heptocarcinoma (HepG2), cervical (HeLa), liver (SMMC-7721), lung (SPC-A1) cancer cells and T cell leukemia (Jurkat) were cultured in the presence of different concentrations of verticillin A and examined for the efforts of verticillin A on the growth rate of these tumor cells in vitro. Verticillin A significantly inhibited the growth of all these tumor cells (Table 1). More importantly, verticillin A inhibited the growth of these tumor cells with concentrations at the nmole level with IC₅₀ from 30 to 122 nM (Table 1), suggesting that verticillin is potentially a potent tumor suppressor

TABLE 1
IC₅₀ of Verticillin A for multiple types of tumor cell lines

| Cancer | Cell line | IC₅₀ (nM)* |
|---|---|---|
| Mammary Carcinoma | Bcap-37 | 119 |
| | MCF-7 | 64 |
| Cervical Cancer | Hela | 122 |
| Hepatoma | SMMC-7721 | 80 |
| | HepG2 | 62 |
| Lung Carcinoma | SPC-A1 | 30 |
| T Cell Leukemia | Jurkat | 37 |

*IC₅₀ was determined by MTT assay

### Example 4

### Verticillin A is a Potent Apoptosis Sensitizer that Overcomes TRAIL and Fas Resistance in Colon Carcinoma Cells

Materials and Methods

Cell Lines

All cell lines used in this study were obtained from American Type Culture Collection (Mannassas, Va.). Cells were maintained in DMEM or RPMI medium (Invitrogen, Carlsbad, Calif.) supplemented with 10% (v/v) fetal bovine serum (FBS), in 37° C. humidified 5% CO₂ incubator.

Apoptosis Assays

Cells were seeded in wells of 96-well plates for 2 days and then treated with different concentrations of mushroom extract fractions or purified verticillin A for 2 days. The cells were stained with propidium iodide (PI) (Trevigen, Gaithersburg, Md.) or PI plus annexin V-Alex Fluor 647 (Biolegend, San Diego, Calif.) and analyzed by flow cytometry.

TRAIL, FasL and TRAIL Receptor Antibody

Recombinant TRAIL protein was expressed and purified from E. coli. MegaFasL was provided by TopoTarget A/S. TRAIL receptor mAb was obtained from Biolegend Inc.

Statistical Analysis

Where indicated, data were represented as the mean±SD. Statistical analysis was carried out using two-sided t test, with p-values<0.05 considered statistically significant.

Results

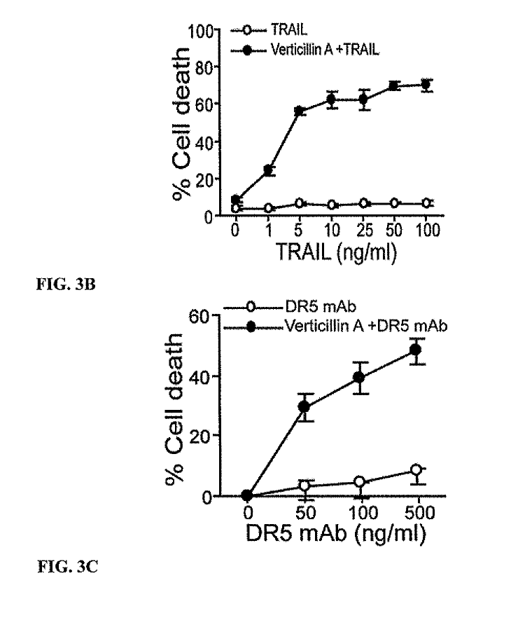

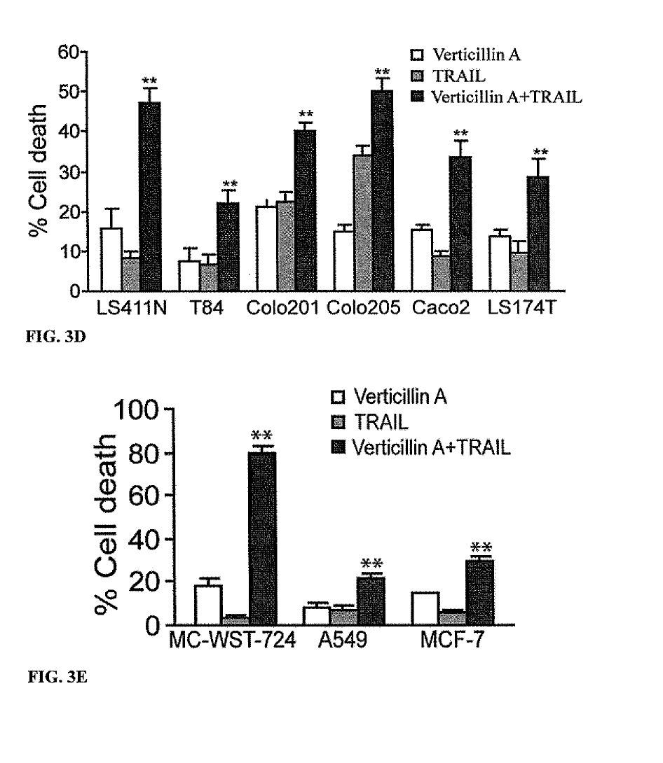

In an experiment to compare the effects of verticillin A and TRAIL on induction of colon carcinoma cell apoptosis, it was surprisingly observed that verticillin A overcomes TRAIL resistance of the metastatic colon carcinoma cell line SW620 (FIG. 3B). SW620 is a metastatic human colon carcinoma cell line that is resistant to TRAIL-induced apoptosis (Voelkel-Johnson C, et al. Mol Cancer Ther 2005 4(9):1320-7). Verticillin A induced significant apoptosis of SW620 cells in a concentration-dependent manner and TRAIL exhibited no apoptosis induction activity in SW620 cells (FIG. 3A-3B). However, when used in combination, very low concentration of verticillin A (10 nM) dramatically sensitized SW620 cells to TRAIL-induced apoptosis (FIG. 3B).

The sensitization effect of verticillin A was also observed in 6 other human colon carcinoma cells (FIG. 3D-3E). The sensitization effects of verticillin A was examined in other types of tumor cells. Co-treatment of sarcoma (MC-WST-724), ovarian carcinoma (A549) and mammary carcinoma (MCF-7) with verticillin A and TRAIL also significantly increased the apoptosis rate than the single agent alone (FIG. 3D-3E).

Analysis of tumor cell cytochrome C release, a biochemical marker of apoptosis, indicated that combinational treatment of verticillin A and TRAIL dramatically increased cytochrome C release in SW620 cells. These data indicate that verticillin A is a potent TRAIL sensitizer that effectively enhances TRAIL-induced tumor cell apoptosis.

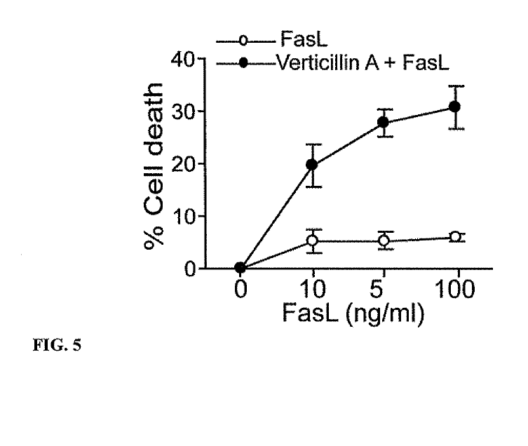

Because the Fas-mediated apoptosis and TRAIL-induced apoptosis share similar signaling pathways, it was next tested whether Verticillin A also sensitizes tumor cells to FasL-induced apoptosis. SW620 cells were treated with verticillin A, followed by treatment with recombinant FasL. It is clear that Verticillin A pre-treatment significantly increases tumor cells to FasL-induced apoptosis (FIG. 5).

### Example 5

### Verticillin A Increases the Efficacy of Etoposide, Cisplatin, 5-FU and Doxorubicin in Suppression of Tumor Cell Growth

Materials and Methods

Cell Lines

All cell lines used in this study were obtained from American Type Culture Collection (Mannassas, Va.). Cells were maintained in DMEM or RPMI medium (Invitrogen, Carlsbad, Calif.) supplemented with 10% (v/v) fetal bovine serum (FBS), in 37° C. humidified 5% CO₂ incubator.

Cell Viability Assays

For cell viability assay, cells were seeded in wells of 96-well plates for 2 days and then treated with different concentrations of mushroom extract fractions or purified verticillin A for 2 days. The cells were then incubated with MTT (3-(4,5-dimethylthiazol-2-yl)-2,5-diphenyltetrazolium bromide) using the MTT assay kit (ATCC, Manassas, Va.) according to the manufacturer's instructions.

Statistical Analysis

Where indicated, data were represented as the mean±SD. Statistical analysis was carried out using two-sided t test, with p-values<0.05 considered statistically significant.

Results

Etoposie, Cisplatin, 5-FU and Doxorubicin are commonly used cancer drugs that suppress tumor cell growth through their cytotoxicity. High cytotoxicity is often associated with high dose of their drugs. Furthermore, tumor cells often develop resistance to these cytotoxic drugs. To determine whether Verticillin A can increase the efficacy of these drugs and overcome drug resistance, the effectiveness of Verticillin A combined with these 4 drugs in suppression of tumor cell growth was tested in vitro. SW620 cell sensitivity to these drugs was first measured. SW620 cells are resistant to Etoposide but sensitive to 5-FU. SW620 cells are only sensitive to high doses of Cisplatin and Doxorubicin (FIG. 6). SW620 cells were next pretreated with Verticillin A, followed by treatment with Etoposide (1 μg/ml), Cisplatin (1 μg/ml), 5-FU (0.1 tag/ml) and Doxorubicin (0.01 μg/ml), respectively. At these lower dose, these drugs exerted minimal inhibitory effects. However, combined these drugs with Verticillin A significantly increased the tumor growth inhibitory effects of these drugs (FIG. 6). Taken together, these data indicate that Verticillin A can be used to 1) overcome tumor cell resistance to these drugs; and 2) reduce the usage dose to decrease the toxicity of these drugs while maintaining the effectiveness.

### Example 6

### Verticillin A Enhances TRAIL-Mediated Tumor Suppression In Vivo

Materials and Methods

In Vivo Tumor Growth Inhibition

Athymic mice were subcutaneously inoculated with human hepatoma HepG2 and colon carcinoma SW620 cells, respectively. The tumor-bearing mice were randomized into experimental groups. The control mice were given saline. The treatment group was intravenously injected with verticillin A at the doses of 1 and 2 mg/kg body weight. Tumor size was measured in 2 diameter with micrometer caliper at the indicate times to permit calculation of tumor volume, V=(a×b²)/2, where “a” is the length and “b” is the width in millimeters. At the end of the experiment, all the animals were sacrificed, The tumor inhibitory rates were calculated using the following formula: [(mean tumor volume of control nude mice−mean tumor volume of treated nude mice)/mean tumor volume of control nude mice]×100%.

Statistical Analysis

Where indicated, data were represented as the mean±SD. Statistical analysis was carried out using two-sided t test, with p-values<0.05 considered statistically significant.

Results

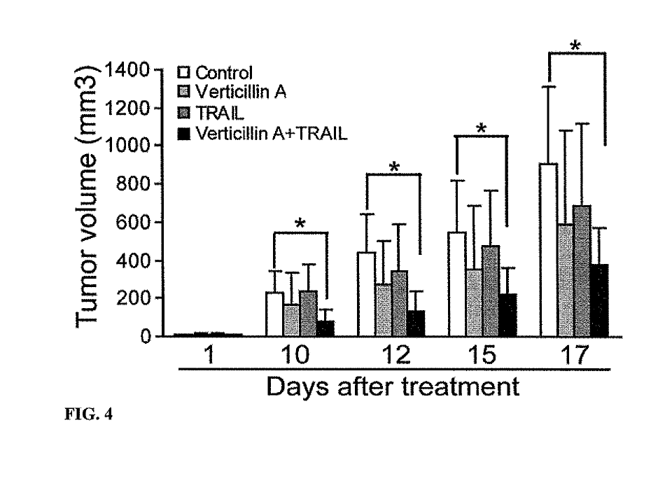

The disclosed observations indicate that verticillin A can dramatically increase TRAIL-induced tumor cell apoptosis. To determine whether these in vitro observations can be extended to enhance TRAIL-mediated tumor suppression in vivo, SW620 cells were injected subcutaneously to athymic mice, verticillin A and TRAIL, either used as single agent or in combination, were then injected to the tumor-bearing mice. Tumor growth rate were measured in different time points. Verticillin A can inhibit tumor cell growth in vitro at high dose and sensitize tumor cells to TRAIL-induced apoptosis at a low dose. To differentiate the role of verticillin A in TRAIL sensitization from its direct tumor growth inhibitory activity, a lower dose (0.125 mg/kg body weight) of verticillin A was used. At this low dose, verticillin A did not exhibit significant tumor suppression activity (FIG. 4). As expected, SW620 tumors are resistant to TRAIL (Voelkel-Johnson C, et al. Mol Cancer Ther 2005 4(9):1320-7). However, combination treatment of low dose of verticillin A and TRAIL significantly inhibited the tumor xenograft growth (FIG. 4). Taken together, these data indicate that verticillin A can effectively overcome TRAIL resistance in TRAIL therapy against metastatic human colon carcinoma.

### Example 7

### Verticillin A Sensitizes Tumor Cells to TRAIL-Mediated Apoptosis Through Repressing Acid Ceramidase (A-CDase)

Materials and Methods

Cell Lines

All cell lines used in this study were obtained from American Type Culture Collection (Mannassas, Va.). Cells were maintained in DMEM or RPMI medium (Invitrogen, Carlsbad, Calif.) supplemented with 10% (v/v) fetal bovine serum (FBS), in 37° C. humidified 5% CO₂ incubator.

### Synthesis of LCL85

(1R,2R)-2_N-[16-(1′-pyridinium)-hexadecanoylamino)-1-(4′-nitrophenyl)-1,3-propandiol bromide, was synthesized by Lipidomics Shared Resource at Medical University of South Carolina, as previously described (Szulc Z M, et al. Bioorg Med Chem 2008 16(2):1015-31).

Gene Silencing

Scramble sRNA (Dharmacon, Lafayette, Colo.) and siRNA specific for human acid ceramidase (A-CDase) (Santa Cruz, Cat#sc-105032) were used to transiently transfect the tumor cells using Lipofectamine 2000 (invitrogen) for approximately 24 h. The tumor cells were then divided into two halves. One half was used to analyze A-CDase mRNA level as previously described (Yang D, et al. Clin Cancer Res 2007 13(17):5202-10). The other half was analyzed for the sensitivity to TRAIL-induced apoptosis as previously described (Yang D, et al. Cancer Res 2009 69(3):1080-8).

Western Blot Analysis

Tumor cells were lysed in lysis buffer containing 20 mM HEPES, pH 7.4, 20 mM NaCl, 10% glycerol, 1% Triton X-100, and a protease inhibitor cocktail (Calbiochem, La Jolla, Calif.). Cellular proteins were separated by 12% or 4-20% SDS-PAGE gradient gels, transferred to Immobilon-P membranes (Millipore, Bedford, Mass.) or nitrocellulose (Bio-Rad, Hercules, Calif.), and probed with the following primary antibodies: anti-FLIP (Cell Sinnaling, Danvers, Mass.) at 1:250; anti-survivin (Santa Cruz Biotech) at 1:100; anti-cIAP1 (cell signaling) at 1:250; anti-xIAP (cell Signaling) at 1:500; anti-Bad (Cell Signaling) at 1:1000; anti-Bax (Cell Signaling) at 1:2000; anti-Mcl-1 (Santa Cruz) at 1:100); anti-A-CDase (BD Biosciences) at 1:1000; anti-CytC (BD Biosciences) at 1:1000; and β-actin (Sigma, St Louis, Mo.) at 1:8000. The blots were then washed and incubated with horseradish peroxidase-conjugated anti-goat (Santa Cruz Biotech), anti-mouse or rabbit (Amersham-Pharmacia, Piscataway, N.J.) IgGs. Blots were detected using the ECL Plus Western detection kit (Amersham Pharmacia Biotech).

Statistical Analysis

Where indicated, data were represented as the mean±SD. Statistical analysis was carried out using two-sided t test, with p-values<0.05 considered statistically significant.

Results

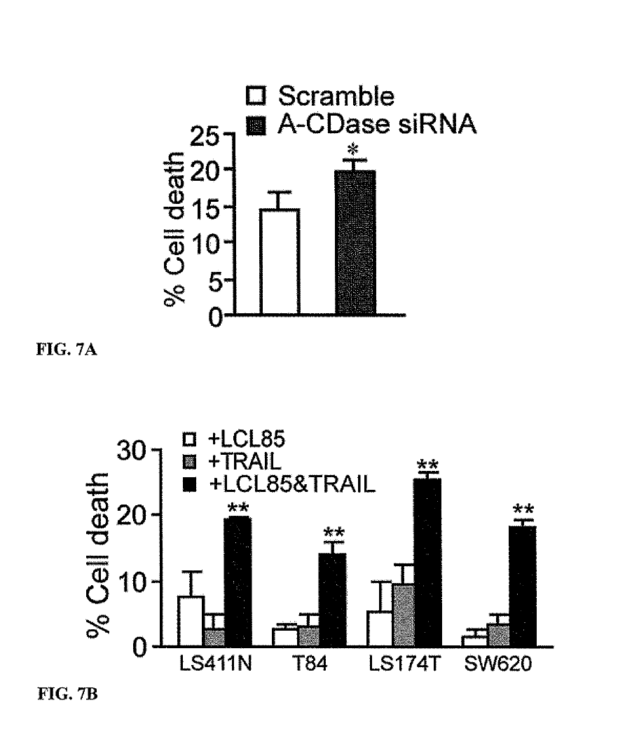

Verticillin A induces DNA fragmentation and cytochrome C release (FIG. 3A), indicating that verticillin A mediates the mitochondrion-dependent apoptosis. To elucidate the molecular mechanisms underlying verticillin A sensitization of tumor cells to TRAIL-mediated apoptosis, the protein levels of genes known to function in the mitochondrion-mediated apoptosis pathway were analyzed. One the multiple protein analyzed was A-CDase, whose expression level was decreased after verticillin A treatment at a dose-dependent manner. To functionally validate the role of A-CDase in TRAIL-resistance in metastatic colon carcinoma cells, A-CDase-specific siRNA was used to silence A-CDase in SW620 cells and the sensitivity of SW620 cells to TRAIL-induced apoptosis was examined. It is clear that silencing A-CDase significantly increased SW620 cell sensitivity to TRAIL-induced apoptosis (FIG. 7A).

A complementary approach was examined to determine the role of A-CDase in TRAIL resistance. Colon carcinoma (LS114N, T84, LS174T and SW620) were treated with A-CDase inhibitor LCL85 prior to TRAIL treatment and analyzed for apoptosis. LCL85 pre-treatment significantly increased the sensitivity of tumor cells to TRAIL-induced apoptosis (FIG. 7B).

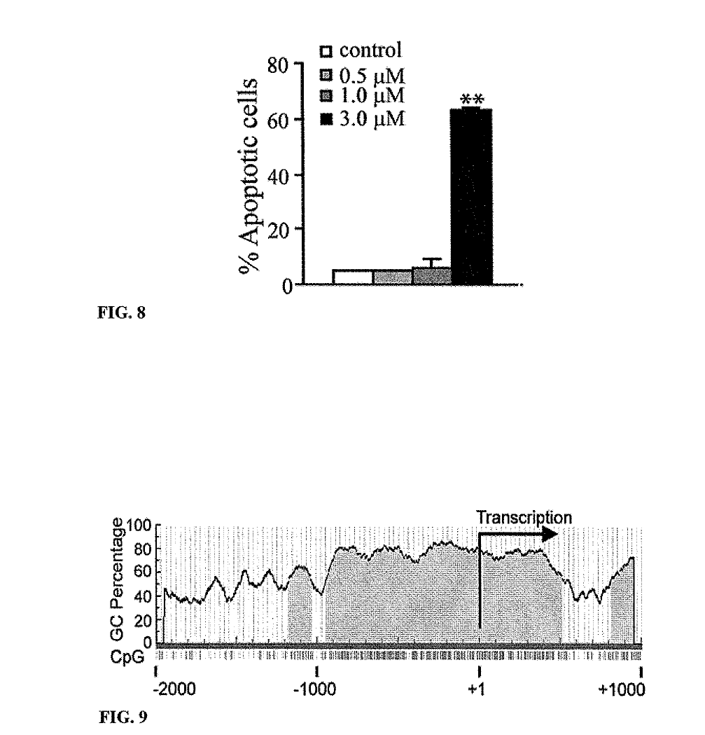

A-CDase catalyzes ceramide degradation to decrease ceramide level in the cells and overexpression of A-Cdase is often associated with apoptosis resistance in tumor cells (Elojeimy S, et al. Mol Ther 2007 15(7):1259-63; Liu X, et al. Front Biosci 2008 13:2293-8; Mao C, et al. Biochim Biophys Acta 2008 1781(9):424-34; Ogretmen B. FEBS Lett 2006 580(23):5467-76; Baran Y, et al. J Biol Chem 2007 282(15):10922-34). Inhibition of A-CDase activity is often associated with increased ceramide level and enhanced apoptosis sensitivity (Liu X, et al. Front Biosci 2008 13:2293-8; Ogretmen B. FEBS Lett 2006 580(23):5467-76; White-Gilbertson S, et al. Oncogene 2009 28(8):1132-41). Next, we examined the effect of exogenous ceramide on tumor cell sensitivity to TRAIL-mediated apoptosis. SW620 cells were incubated with different concentrations of C16 ceramide for 2 h and then treated with TRAIL. C16 ceramide exhibited apoptosis-inducing activity at a dose-dependent manner. However, C16 pretreatment dramatically increased the tumor cell sensitivity to TRAIL-mediated apoptosis (FIG. 8). Taken together, these data indicate verticillin A sensitizes tumor cells to TRAIL-mediated apoptosis in vitro and enhances TRAIL-mediated tumor growth suppression in vivo at least partially through repressing A-CDase.

### Example 8

### Verticillin A Regulates BNIP3 Expression in Heptocarcinoma and Colon Carcinoma Cells

Because verticillin A enhances tumor cell apoptosis through the mitochondrion-dependent pathway, the expression levels of apoptosis-related genes of the mitochondrion-mediated apoptosis pathway was analyzed. BNIP 3, a Bcl-2 family protein with known pro-apoptotic function, was identified as a target of verticillin A. Verticillin A increased BNIP3 expression level in a dose-dependent manner in SW620 and HepG2 cells.

### Example 9

### Verticillin a Inhibits BNIP3 Promoter DNA Methylation to Activate BNIP 3 Expression

Analysis of the human BNIP3 promoter sequence revealed that there are CpG islands in the BNIP3 promoter region (FIG. 9). To determine whether the BNIP3 promoter DNA is hypermethylated, MS-PCR techniques were used to analyze the BNIP3 promoter DNA methylation status in 5 human colon carcinoma specimens (4 Liver metastases: 4516A4(LM), 25033A3F (LM), 27340A3PB(LM), 4793A5(LM) and 1 primary adenocarcinoma: 072694(P)), 3 human colon carcinoma cell lines (HCT116, LS411N, SW620), and human Hepatoma cell line HepG2 cells.

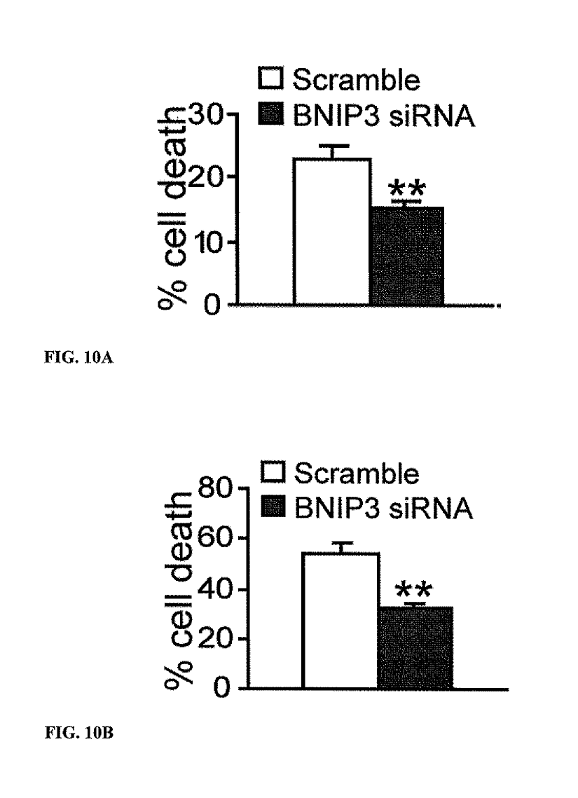

MS-PCR analysis revealed that the BNIP promoter DNA is methylated in all 5 tumor specimens and all 4 human tumor cell lines. More importantly, silencing BNIP3 expression significantly decreased tumor cell sensitivity to Verticillin A-induced or Verticillin A-sensitized and TRAIL-induced apoptosis (FIG. 10).

To determine whether verticillin A inhibits DNA methylation in other gene promoters, 4 other genes (IRF8, Neurog1, p15ink4b, RUNX3) with known promoter DNA methylation in human colon carcinoma cells were selected. SW620 cells were treated with AzadC (0-1.0 μM) and verticillin A (0-50 nM), respectively, and analyzed for the expression of BNIP3 and these 4 genes. As expected, the expression levels of BNIP3 and these 4 genes were dramatically increased after azadC treatment. However, verticillin A treatment also dramatically increased the expression levels of BNIP3 and these 4 genes. Taken together, these data indicate that verticillin A is a general DNA methylation inhibitor in human colon and heptocarcinoma cells.

### Example 10

### Verticillin A is a Small Molecule TET1 Activator

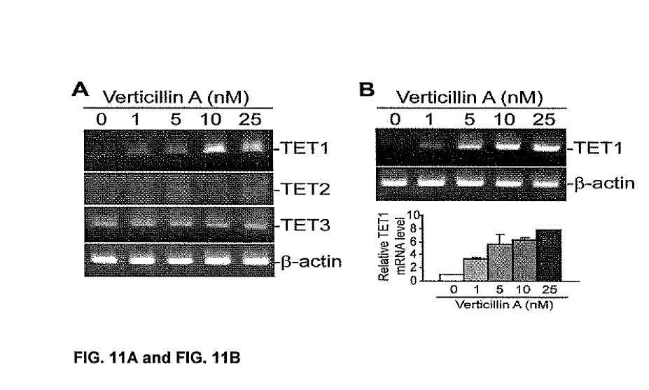

TET1 is an enzyme with very recently identified function in converting 5-methylcytosine (5mC) to 5-hydroxylmethylcytosine (5hmC), and eventually to unmodified Cytosine to reverse DNA methylation (Veron N, Peters A H. Epigenetics: Tet proteins in the limelight. Nature 2011; 473:293-4; Ito et al. Tet proteins can convert 5-methylcytosine to 5-formylcytosine and 5-carboxylcytosine. Science 2011; 333:1300-3; Guo et al. Hydroxylation of 5-methylcytosine by TET1 promotes active DNA demethylation in the adult brain. Cell 2011; 145:423-34). Verticillin A up-regulates TET1 expression in colon carcinoma cells in a dose-dependent manner (FIG. 11). Thus, Verticillin A is a small molecule TET1 activator. Verticillin A appears to be the first reported small molecule TET1 activator.

TET1 is currently at the center of DNA methylation research in stem cells. The findings that Verticillin A up-regulates TET1 expression indicates that Verticillin A is useful as a DNA demethylating agent for cancer therapy.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A composition comprising an epidithiodioxopiprazine defined by the following structure: 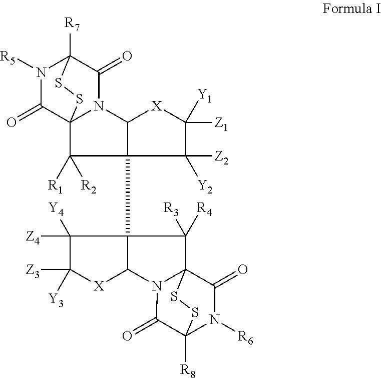
wherein R₁-R₄, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or
R₁ is absent and R₂ may be a ketone or a substituted or unsubstituted exocyclic alkylene group, and/or R₃ is absent and R₄ may be a ketone or a substituted or unsubstituted exocyclic alkylene group;
R₅-R₈, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;
Y₁-Y₄ and Z₁-Z₄, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
Y₁ and Y₂, Z₁ and Z₂, Y₃ and Y₄, and/or Z₃ and Z₄, taken together with the atoms to which they are attached, may be C₃-C₂₀ cyclic, substituted C₃-C₂₀ cyclic, heterocyclic, or substituted heterocyclic group; or
Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; or
Z₃ and Z₄ are absent and Y₃ and Y₄, taken together with the atoms to which they are attached may be a π-bond, phenyl, substituted phenyl, aryl, substituted aryl, heteroaryl, substituted heteroaryl, polyaryl, or substituted polyaryl; or
Z₁ is absent and Y₁ may be a ketone or a substituted or unsubstituted exocyclic alkylene group, Z₂ is absent and Y₂ may be a ketone or a substituted or unsubstituted exocyclic alkylene group, Z₃ is absent and Y₃ may be a ketone or a substituted or unsubstituted exocyclic alkylene group, and/or Z₄ is absent and Y₄ may be a ketone or a substituted or unsubstituted exocyclic alkylene group; and
X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;
or a pharmaceutically acceptable salt thereof,
wherein the epidithiodioxopiprazine is in an amount effective to sensitize a target cell to TRAIL-induced apoptosis.

2. The composition of claim 1, wherein the epidithiodioxopiprazine is selected from the group consisting of Verticillin A, Verticillin B, Verticillin D, Verticillin E, Verticillin F, 11-deoxyverticillin, 11,11′-dideoxyverticillin, Chaetocin, Gliotoxin, and Chaetomin.

3. The composition of claim 1, further comprising a death receptor agonist.

4. The composition of claim 3, wherein the death receptor agonist is TNF-related apoptosis-inducing ligand (TRAIL).

5. The composition of claim 4, wherein the death receptor agonist is an antibody that selectively binds and activates DR4 (TRAIL-R1) or DR5 (TRAIL-R2).

6. A method of inducing apoptosis in a target cell, comprising:
contacting the target cell with a first composition comprising an effective amount of an epidithiodioxopiprazine defined by the following structure: 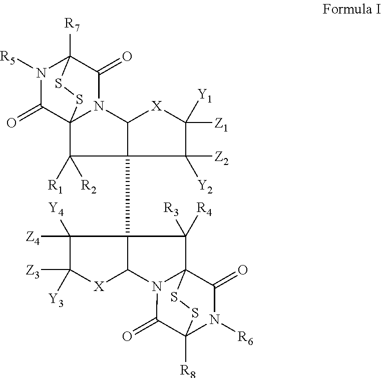
wherein R₁-R₄, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or
R₁ is absent and R₂ may be a ketone or a substituted or unsubstituted exocyclic alkylene group, and/or R₃ is absent and R₄ may be a ketone or a substituted or unsubstituted exocyclic alkylene group;
R₅-R₈, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;
Y₁-Y₄ and Z₁-Z₄, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
Y₁ and Y₂, Z₁ and Z₂, Y₃ and Y₄, and/or Z₃ and Z₄, taken together with the atoms to which they are attached, may be C₃-C₂₀ cyclic, substituted C₃-C₂₀ cyclic, heterocyclic, or substituted heterocyclic group; or
Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; or
Z₃ and Z₄ are absent and Y₃ and Y₄, taken together with the atoms to which they are attached may be a π-bond, phenyl, substituted phenyl, aryl, substituted aryl, heteroaryl, substituted heteroaryl, polyaryl, or substituted polyaryl; or
Z₁ is absent and Y₁ may be a ketone or a substituted or unsubstituted exocyclic alkylene group, Z₂ is absent and Y₂ may be a ketone or a substituted or unsubstituted exocyclic alkylene group, Z₃ is absent and Y₃ may be a ketone or a substituted or unsubstituted exocyclic alkylene group, and/or Z₄ is absent and Y₄ may be a ketone or a substituted or unsubstituted exocyclic alkylene group; and
X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;
or a pharmaceutically acceptable salt thereof; and
contacting the target cell with a second composition comprising an effective amount of death receptor agonist.

7. The method of claim 6, wherein the epidithiodioxopiprazine is selected from the group consisting of Verticillin A, Verticillin 13, Verticillin D, Verticillin E, Verticillin F, 11-deoxyverticillin, 11,11′-dideoxyverticillin, Chaetocin, Gliotoxin, and Chaetomin.

8. The method of claim 6, wherein the target cell is contacted with the first composition from about 4 hours to about 24 hours before the second composition.

9. The method of claim 6, wherein the target cell is contacted with the first composition within 4 hours before the second composition.

10. The method of claim 6, wherein the target cell expresses DR4 (TRAIL-R1) or DR5 (TRAIL-R2).

11. The method of claim 10, wherein the target cell is a cancer cell.

12. The method of claim 10, wherein the target cell is a tumor cell.

13. The method of claim 10, wherein the target cell is resistant to TNF-related apoptosis-inducing ligand (TRAIL)-induced apoptosis.

14. A method of treating cancer in a subject in need thereof, comprising administering to the subject a composition comprising a therapeutically effective amount a death receptor agonist and a composition comprising a therapeutically effective amount of an epidithiodioxopiprazine defined by the following structure: 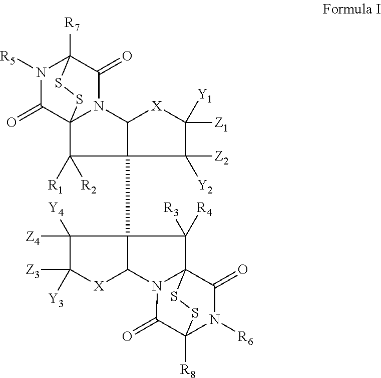
wherein R₁-R₄, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or
R₁ is absent and R₂ may be a ketone or a substituted or unsubstituted exocyclic alkylene group, and/or R₃ is absent and R₄ may be a ketone or a substituted or unsubstituted exocyclic alkylene group;
R₅-R₈, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;
Y₁-Y₄ and Z₁-Z₄, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
Y₁ and Y₂, Z₁ and Z₂, Y₃ and Y₄, and/or Z₃ and Z₄, taken together with the atoms to which they are attached, may be C₃-C₂₀ cyclic, substituted C₃-C₂₀ cyclic, heterocyclic, or substituted heterocyclic group; or
Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; or
Z₃ and Z₄ are absent and Y₃ and Y₄, taken together with the atoms to which they are attached may be a π-bond, phenyl, substituted phenyl, aryl, substituted aryl, heteroaryl, substituted heteroaryl, polyaryl, or substituted polyaryl; or
Z₁ is absent and Y₁ may be a ketone or a substituted or unsubstituted exocyclic alkylene group, Z₂ is absent and Y₂ may be a ketone or a substituted or unsubstituted exocyclic alkylene group, Z₃ is absent and Y₃ may be a ketone or a substituted or unsubstituted exocyclic alkylene group, and/or Z₄ is absent and Y₄ may be a ketone or a substituted or unsubstituted exocyclic alkylene group; and
X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;
or a pharmaceutically acceptable salt thereof.

15. The method of claim 14, wherein the death receptor agonist and the epidithiodioxopiprazine are in the same composition.

16. A method of sensitizing a cancer to apoptosis induced by a death receptor agonist in a subject in need thereof, comprising administering to the subject a composition comprising a therapeutically effective amount of an epidithiodioxopiprazine defined by the following structure: 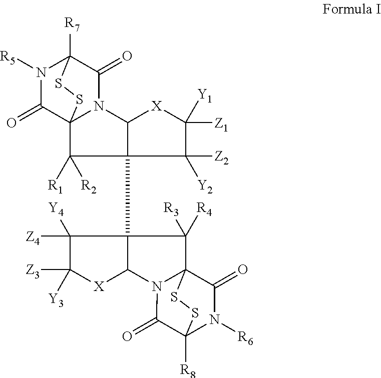
wherein R₁-R₄, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or
R₁ is absent and R₂ may be a ketone or a substituted or unsubstituted exocyclic alkylene group, and/or R₃ is absent and R₄ may be a ketone or a substituted or unsubstituted exocyclic alkylene group;
R₅-R₈, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;
Y₁-Y₄ and Z₁-Z₄, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
Y₁ and Y₂, Z₁ and Z₂, Y₃ and Y₄, and/or Z₃ and Z₄, taken together with the atoms to which they are attached, may be C₃-C₂₀ cyclic, substituted C₃-C₂₀ cyclic, heterocyclic, or substituted heterocyclic group; or
Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; or
Z₃ and Z₄ are absent and Y₃ and Y₄, taken together with the atoms to which they are attached may be a π-bond, phenyl, substituted phenyl, aryl, substituted aryl, heteroaryl, substituted heteroaryl, polyaryl, or substituted polyaryl; or
Z₁ is absent and Y₁ may be a ketone or a substituted or unsubstituted exocyclic alkylene group, Z₂ is absent and Y₂ may be a ketone or a substituted or unsubstituted exocyclic alkylene group, Z₃ is absent and Y₃ may be a ketone or a substituted or unsubstituted exocyclic alkylene group, and/or Z₄ is absent and Y₄ may be a ketone or a substituted or unsubstituted exocyclic alkylene group; and
X, taken independently, is a substituted or unsubstituted carbon atom, or a heteroatom such as —O—, —NR—, —S—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;
or a pharmaceutically acceptable salt thereof;
wherein the epidithiodioxopiprazine is administered in an amount effective to sensitize the cancer to apoptosis induced by the death receptor agonist.

17. The method of claim 16, wherein the composition comprising epidithiodioxopiprazine is administered from about 4 hours to about 24 hours before the death receptor agonist.

18. The method of claim 14, wherein the epidithiodioxopiprazine is selected from the group consisting of Verticillin A, Verticillin B, Verticillin D, Verticillin E, Verticillin F, 11-deoxyverticillin, 11,11′-dideoxyverticillin, Chaetocin, Gliotoxin, and Chaetomin.

19. The method of claim 14, wherein the cancer is resistant to TNF-related apoptosis-inducing ligand (TRAIL) treatment.

20. The method of claim 6, wherein the death receptor agonist is TNF-related apoptosis-inducing ligand (TRAIL).

21. The method of any one of claim 6, wherein the death receptor agonist is an antibody that selectively binds and activates DR4 (TRAIL-R1) or DR5 (TRAIL-R2).

22. A method of reducing cancer resistance to an antineoplastic drug in a subject in need thereof, comprising administering to the subject a therapeutically effective amount of an epidithiodioxopiprazine defined by the following structure: 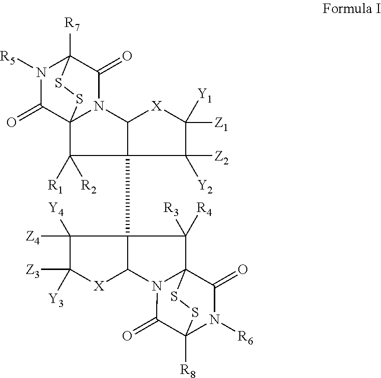
wherein R₁-R₄, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₁-R₄ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
R₁ and R₂ and/or R₃ and R₄ taken together with the atom to which they are attached may be a 1-8 membered substituted or unsubstituted non-aromatic carbocyclic or heterocyclic ring, i.e. including at least one sp³ hybridized atom, and preferably a plurality of sp³ hybridized atoms; or
R₁ is absent and R₂ may be a ketone or a substituted or unsubstituted exocyclic alkylene group, and/or R₃ is absent and R₄ may be a ketone or a substituted or unsubstituted exocyclic alkylene group;
R₅-R₈, taken independently, may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group;
Y₁-Y₄ and Z₁-Z₄, taken independently may be a hydrogen atom, a halogen atom, a hydroxyl group, or any other organic groupings containing any number of carbon atoms, preferably 1-14 carbon atoms, and optionally include one or more heteroatoms such as oxygen, sulfur, or nitrogen grouping in linear, branched, or cyclic structural formats, representative R₅-R₈ groupings being alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, or polypeptide group; or
Y₁ and Y₂, Z₁ and Z₂, Y₃ and Y₄, and/or Z₃ and Z₄, taken together with the atoms to which they are attached, may be C₃-C₂₀ cyclic, substituted C₃-C₂₀ cyclic, heterocyclic, or substituted heterocyclic group; or
Z₁ and Z₂ are absent and Y₁ and Y₂, taken together with the atoms to which they are attached may be a π-bond, phenyl, substituted phenyl, aryl, substituted aryl, heteroaryl, substituted heteroaryl, polyaryl, or substituted polyaryl; or
Z₃ and Z₄ are absent and Y₃ and Y₄, taken together with the atoms to which they are attached may be a π-bond, phenyl, substituted phenyl, aryl, substituted aryl, heteroaryl, substituted heteroaryl, polyaryl, or substituted polyaryl; or
Z₁ is absent and Y₁ may be a ketone or a substituted or unsubstituted exocyclic alkylene group, Z₂ is absent and Y₂ may be a ketone or a substituted or unsubstituted exocyclic alkylene group, Z₃ is absent and Y₃ may be a ketone or a substituted or unsubstituted exocyclic alkylene group, and/or Z₄ is absent and Y₄ may be a ketone or a substituted or unsubstituted exocyclic alkylene group; and
X, taken independently, is a substituted or unsubstituted carbon atom, or a heteroatom such as —O—, —NR—, or —Se—, wherein R may be a hydrogen atom or an alkyl, substituted alkyl, alkenyl, substituted alkenyl, alkynyl, substituted alkynyl, phenyl, substituted phenyl, aryl, substituted aryl, heteroaryl, substituted heteroaryl group;
or a pharmaceutically acceptable salt thereof;
wherein the epidithiodioxopiprazine is administered in an amount effective to sensitize the cancer to apoptosis induced by the antineoplastic drug.

23. The method of claim 22, wherein the epidithiodioxopiprazine is selected from the group consisting of Verticillin A, Verticillin B, Verticillin D, Verticillin E, Verticillin F, 11-deoxyverticillin, 11,11′-dideoxyverticillin, Chaetocin, Gliotoxin, and Chaetomin.

24. The method of claim 22, wherein the epidithiodioxopiprazine is administered from about 4 hours to about 24 hours before the antineoplastic drug.

25. The method of claim 22, wherein the antineoplastic drug is selected from the group consisting of etoposide, cisplatin, 5-FU, and doxorubicin.
